(19) United States
(12) Patent Application Publication
(10) Pub. No.: US 2013/0101509 A1
(43) Pub. Date: Apr. 25, 2013

(54) IMAGING AND RADIOTHERAPY METHODS

(71) Applicants: GE HEALTHCARE LIMITED, LITTLE CHALFONT (GB); GENERAL ELECTRIC COMPANY, SCHENECTADY, NY (US)

(72) Inventors: ALAN CUTHBERTSON, OSLO (NO); PETER BRIAN IVESON, AMERSHAM (GB); RAJIV BHALLA, AMERSHAM (GB); VIJAYA RAJ KUNIYIL KULANGARA, BANGALORE (IN)

(73) Assignees: GENERAL ELECTRIC COMPANY, SCHENECTADY, NY (US); GE HEALTHCARE LIMITED, LITTLE CHALFONT (GB)

(21) Appl. No.: 13/622,483

(22) Filed: Sep. 19, 2012

Related U.S. Application Data
(63) Continuation-in-part of application No. 13/124,703, filed on Apr. 18, 2011.
Application No. 13/124,703 is a division of application No. 61/107,001, filed on Oct. 21, 2008.

(30) Foreign Application Priority Data
Oct. 21, 2008 (GB) 0819280.9

(51) Int. Cl. A61K 51/04; C07B 59/00; C07C 47/575

(52) U.S. Cl. CPC A61K 51/0497; C07C 47/575; C07B 59/001
USPC 424/1.89; 568/441

(57) ABSTRACT

The present invention relates to in vivo imaging and radiotherapeutic methods and agents which target the enzyme aldehyde dehydrogenase (ALDH) and that are suitable for the in vivo imaging of tumours and treatment of cancer.

The present invention relates to in vivo imaging and radiotherapeutic methods and agents suitable for the in vivo imaging of tumours and treatment of cancer. It further relates to methods and agents which target the enzyme aldehyde dehydrogenase (ALDH). The agents have utility for in vivo imaging by Positron Emission Tomography (PET), Single Photon Emission Computed Tomography (SPECT) imaging, Optical Imaging (OI) and radiotherapy (RT).

Recently the stem cell model of cancer has emerged based on the principle that a sub-population of tumour initiating cells are present in the tumour which are distinct from the bulk cells of the tumour. The model predicts that eradication of the bulk of the tumour cells by chemotherapy or radiotherapy will at best result in temporary remission if cancer stem cells are left behind following treatment. It is also known that these stem cell-like populations are more resistant to many of the alkylating agents used in standard chemotherapy regimes [Gordon, M. Y., et al., Leuk. Res. 9, 1017, 1985]. For example, clinical studies have shown the benefit of purging samples with 4-hydroperoxycyclophosphamide (4-HC) before autologous bone marrow transplantation (ABMT) which removes committed progenitor cells but leaves the stem cell population largely intact [Kaizer, H., et al., Blood, 65, 1504-1510, 1985]. In addition, breast cancer studies have demonstrated correlation between ALDH expression in tumour tissue and poor clinical outcome and have also suggested ALDH as a marker of malignant mammary stem cells [Ginestier, C., et al., Cell Stem Cell, 1, 555, 2007].

Interestingly, the differential sensitivities of stem cells to 4-HC has been demonstrated to correlate with the intracellular activities of the enzyme aldehyde dehydrogenase [Sahovic, E. A. et al., Cancer Research, 48, 1223-1226, 1988]. Enzyme systems such as aldehyde dehydrogenase (ALDH) are ideal targets. The number of cancer stem cells is small in relation to the total tumour composition and more traditional approach employing 1:1 receptor targeting may therefore have limited value in molecular imaging and RT applications. However an imaging or therapeutic dose may be obtained within the stem cell population if the agent accumulates specifically within the stem cells. This signal amplification effect can be achieved by employing substrates for ALDH which freely diffuse through the tumour mass, are efficiently converted by the enzyme inside the cell from an aldehyde to a polar carboxylic acid which is trapped preferentially within the stem cell. Fluorescent substrates for ALDH are known and are typically used for the in vitro separation of stem cell populations from complex cellular mixtures. WO96/36344 provides examples of dansylaminoacetaldehyde derivatives and WO2008/036419 teaches a method for detecting ALDH activity in cancer tissue samples using the BODIPY dye substrate ALDEFLUOR. In both cases the dyes are taken up by stem cells and processed by ALDH to give a negatively charged dye which accumulates intracellularly in the stem cell. The cells are then be sorted by flow cytometry.

However, there still exists a need in oncology for in vivo imaging methods capable of distinguishing the cancer stem cell population to provide valuable prognostic, diagnostic and therapy monitoring information. In addition cancer stem cell targeted agents carrying therapeutic radionuclides such as iodine-131 may deliver a therapeutic payload directly to the stem cell, thus enhancing the benefit of therapy.

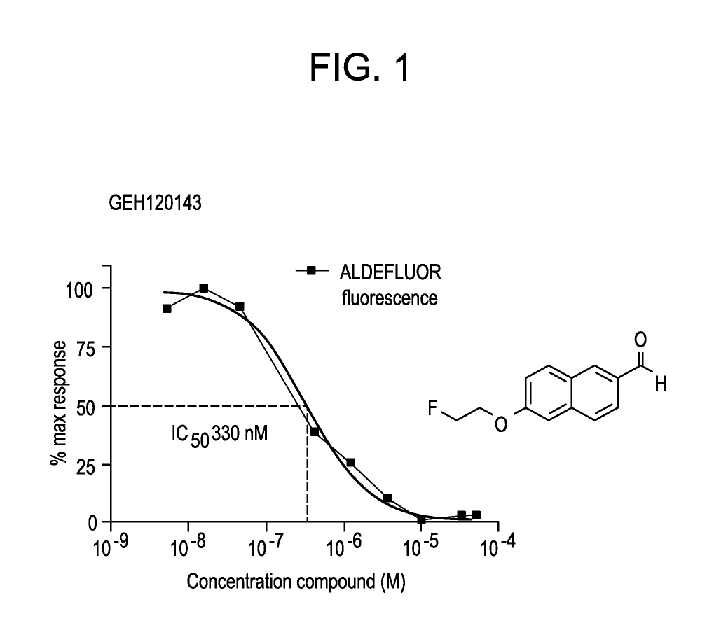

FIG. 1 shows the relationship between the concentration of GEH120143 and response as measured by Aldefluor fluorescence. Also illustrated is the IC₅₀ value.

According to a first aspect of the invention, there is provided a method for detection of tumour stem cells in a subject, comprising:

(i) administrating a detectably labelled substrate for ALDH to said subject;
(ii) detecting uptake of said detectably labelled substrate for ALDH by in vivo imaging.

The “detectably labelled substrate for ALDH” is a substrate for ALDH which preferably has no other known biological activity, and is suitably a compound of formula (I):

A-(B)ₙ—C(O)H (I)

or a salt or solvate thereof, wherein
n is an integer 0 or 1;
A is either a radioimaging moiety or an optical imaging moiety;
B is a carrier moiety; and
the compound of formula (I) has a molecular weight of below 800 Daltons,

The term “radioimaging moiety” means a group comprising (a) a non-metal radiolabel suitable for imaging with PET or SPECT such as ^{123, 124, 122}I, ⁷⁵Br, ⁷⁶Br, ⁷⁷Br, ¹³N, ¹¹C, or ¹⁸F or (b) a chelated radioimaging metal. In one aspect of the invention, the radioimaging moiety comprises a non-metal radiolabel suitable for imaging with PET or SPECT, suitably selected from ^{123, 124, 122}I, ⁷⁵Br, ⁷⁶Br, ⁷⁷Br, ¹³N, ¹¹C, and ¹⁸F, more suitably ^{123, 124, 122}I or ¹⁸F, and is preferably ¹⁸F.

Suitable radioimaging moieties comprising a non-metal radiolabel are known in the art, and typically comprise a C₁₋₃₀hydrocarbyl linker group optionally further containing 1 to 10 heteroatoms selected from nitrogen, oxygen, and sulphur and having the non-metal radiolabel covalently attached thereto or incorporated therein or alternatively, in the case of a radiohalo ^{123, 124, 122}I, ⁷⁵Br, ⁷⁶Br, ⁷⁷Br, or ¹⁸F, such a radiolabel may be directly bonded to the rest of the compound of formula (I). Radiohalo radiolabels are commonly incorporated as radiohaloC₁₋₆alkyl groups such as [¹⁸F]fluoroethyl or [¹⁸F]fluoropropyl, radiohaloC₁₋₆alkoxy groups such as [¹⁸F]fluoroethoxy or [¹⁸F]fluoromethoxy. [¹¹C]carbon radiolabels are commonly incorporated as [¹¹C]C₁₋₆alkyl groups such as [¹¹C]methyl or [¹¹C]ethyl or as a [¹¹C]carbonyl group.

Certain reagents are commonly used to introduce an ¹⁸F radiolabel which include N-succinimidyl-4-[¹⁸F]fluorobenzoate, m-maleimido-N-(p-[¹⁸F]fluorobenzyl)benzamide, N-(p-[¹⁸F]fluorophenyl)maleimide, and 4-[¹⁸F]fluorophenacylbromide and are reviewed for example in Okarvi, European Journal of Nuclear Medicine 28, (7), 2001. Further description of prosthetic groups and methods for incorporating them into a ligand may be found in published international patent applications WO03/080544, WO2004/080492, and WO2006/067376.

When radioimaging moiety A comprises a chelated radioimaging metal, it comprises a chelating group as defined below and a radioimaging metal. The chelating group may be directly bonded to the rest of the compound of formula (I) or may be attached by way of a C₁₋₃₀hydrocarbyl linker group optionally further containing 1 to 10 heteroatoms selected from nitrogen, oxygen, and sulphur which serves to space the chelate sterically from the rest of the compound. As used herein, the term “radioimaging metal” means either a positron emitter such as ⁶⁴Cu, ⁴⁸V, ⁵²Fe, ⁵⁵Co, ^{94m}Tc ⁶⁸Gd, or ⁶⁸Ga; or a gamma-emitter such as ^{99m}Tc, ¹¹¹In, ^{113m}In, ⁶⁷Gd, or ⁶⁷Ga. Preferred radioimaging metals are selected from ^{99m}Tc, ⁶⁴Cu, ⁶⁸Ga and ¹¹¹In. In one aspect, the radioimaging metal is a gamma-emitter, especially ^{99m}Tc. In all cases, the radioimaging metal is chelated to a chelating group as defined below.

The term “optical imaging moiety” means a fluorescent dye or chromophore which is capable of detection either directly or indirectly in an optical imaging procedure using light of green to near-infrared wavelength (500-1200 nm, preferably 600-1000 nm) and is either directly bonded to the rest of the compound of formula (I) or is attached by way of a C₁₋₃₀hydrocarbyl linker group optionally further containing 1 to 10 heteroatoms selected from nitrogen, oxygen, and sulphur. Preferably, the optical imaging moiety has fluorescent properties and is more preferably a fluorescent dye. Since the optical imaging moiety must be suitable for imaging the mammalian body in vivo, it must also be biocompatible. By the term “biocompatible” is meant non-toxic and hence suitable for administration to the mammalian body, especially the human body without adverse reaction, or pain or discomfort on administration.

Suitable optical imaging moieties include groups having an extensive delocalized electron system, for example, cyanines, merocyanines, indocyanines, phthalocyanines, naphthalocyanines, triphenylmethines, porphyrins, pyrilium dyes, thiapyriliup dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots). Preferably, the optical imaging moiety of the present invention does not comprise a metal complex, and is preferably a synthetic organic dye.

Particular examples of optical imaging moieties which may be used include: fluorescein, sulforhodamine 101 (Texas Red), rhodamine B, rhodamine 6G, rhodamine 19, indocyanine green, the cyanine dyes Cy2, Cy3, Cy35, Cy5, Cy5.5, Cy7, Marina Blue, Pacific Blue, Oregon Green 88, Oregon Green 514, tetramethylrhodamine, and Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor®700, and Alexa Fluor® 750.

Suitable salts according to the invention include (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycollic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine.

Suitable solvates according to the invention include those formed with ethanol, water, saline, physiological buffer and glycol.

The term “subject” means a mammal, preferably a human who has or is suspected of having a tumour, especially a solid tumour for example in the breast, colon, prostate, bone, bladder, ovary, pancreas, bowel, lung, kidney, adrenal glands, liver, or skin. Examples of solid tumours include sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumour, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumour, cervical cancer, testicular tumour, lung carcinoma, small cell lung carcinoma, bladder carcinoma, epithelial carcinoma, glioma, astrocytoma, medulloblastoma, craniopharyngioma, endymoma, pinealoma, hemangioblastoma, acoustic neuroma, oligiodendroglioma, meningioma, melanoma, neuroblastoma, and retinoblastoma.

Such a subject may have presented one or more symptoms indicative of a cancer such as a lump or mass, or may be being routinely screened for cancer, or screened for cancer due to presence of one or more risk factors, may have been identified as having cancer, or have had cancer in the past but being screened in remission.

The term “cancer patient” means a mammal, preferably a human, who is being treated for primary or metastatic cancer such as a solid tumour as defined above or a hematologic malignancy (for example acute or chronic myeloid leukaemia). Examples of such cancers include carcinoma, lymphoma, blastoma, sarcoma, and leukaemia.

As used herein the term “halo” either alone or as part of another term means iodo, bromo, chloro, or fluoro.

As used herein the term “alkyl” either alone or as part of another term means a straight, branched or cyclic alkyl group.

As used herein the term “aryl” either alone or as part of another term means a carbocyclic aromatic system, suitable examples being phenyl or naphthyl, more suitably phenyl.

As used herein the term “hydrocarbyl group” means an organic substituent consisting of carbon and hydrogen, such groups may include saturated, unsaturated, or aromatic portions.

Suitable “chelating groups” in group A include those of Formula Z

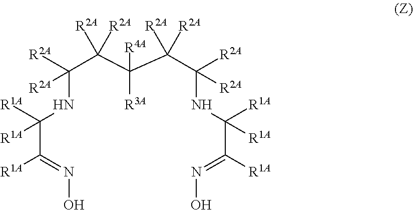

where:
each R^{1A}, R^{2A}, R^{3A} and R^{4A} is independently an R^{A} group; each R^{A} group is independently H or C₁₋₁₀ alkyl, C₃₋₁₀ alkylaryl, C₂₋₁₀ alkoxyalkyl, C₁₋₁₀ hydroxyalkyl, C₁₋₁₀ alkylamine, C₁₋₁₀ fluoroalkyl, or 2 or more R^{A} groups, together with the atoms to which they are attached form a carbocyclic, heterocyclic, saturated or unsaturated ring,
or A can comprise a chelating group given by formula (i), (ii), (iii), or (iv)

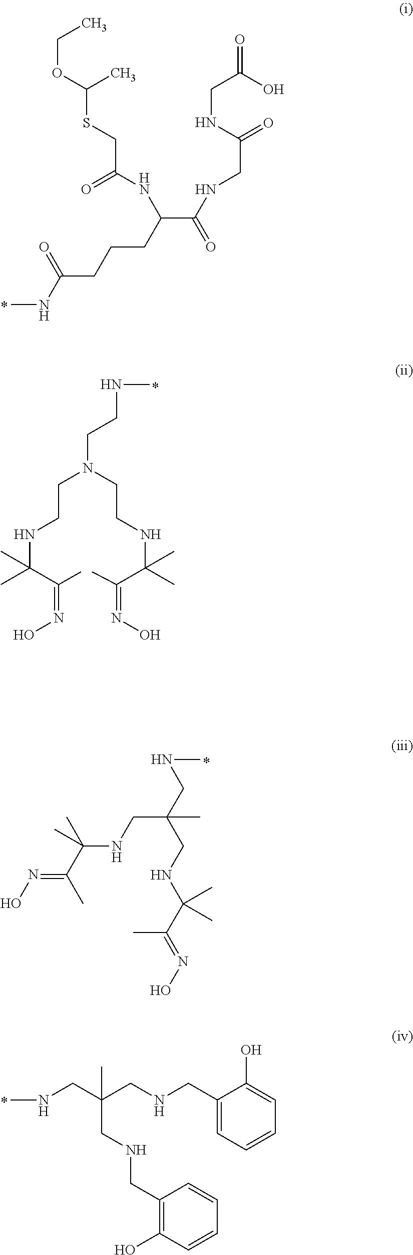

A preferred example of a chelating group is represented by formula (v).

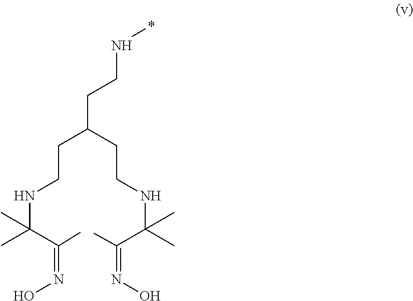 [SMILES: CNCCC(CCNC(C)(C)/C(C)=N\O)CCNC(C)(C)/C(C)=N\O]

Compounds of formula (I) comprising chelating groups of Formula Z can be radiolabelled to give good radiochemical purity (RCP), at room temperature, under aqueous conditions at near neutral pH.

Further suitable chelating groups include:

(i) N₃S chelating groups having a thioltriamide donor set such as MAG₃ (mercaptoacetyltriglycine) and related chelating groups; or having a diamidepyridinethiol donor set such as picolinomide (Pica);
(ii) N₂S₂ chelating groups having a diaminedithiol donor set such as bisaminothiol (BAT) or ethylcysteinate dimer (ECD), or an amideaminedithiol donor set such as monoamine-monoamide (MAMA);
(iii) N₄ chelating groups which are open chain or macrocyclic ligands having a tetramine, amidetriamine or diamidediamine donor set, such as cyclam, monoxocyclam or dioxocyclam; or
(iv) N₂O₂ chelating groups having a diaminediphenol donor set; or
(v) 1,4,7,10-tetraazacyclododecane-N,N′,N″,N′″-tetraacetoc acid (DOTA), 1,4,7-triazacyclononane-N,N′,N″-triacetic acid (NOTA) and derivatives of DOTA and NOTA, for example as described in WO89/001475.

The above described chelating groups (i) to (iv) are particularly suitable for complexing technetium, for example, ^{94m}Tc or ^{99m}Tc, and are described more fully by Jurisson et al [Chem. Rev., 99, 2205-2218 (1999)]. The chelating groups above are also useful for other metals, such as copper (⁶⁴Cu or ⁶⁷Cu), vanadium (for example, ⁴⁸V), iron (for example, ⁵²Fe), or cobalt (for example, ⁵⁵Co). Chelating groups (v) are particularly suitably for complexing Gallium (e.g. ⁶⁷Ga or ⁶⁸Ga). Other suitable ligands are described in Sandoz WO 91/01144, which includes ligands which are particularly suitable for indium, yttrium and gadolinium, especially macrocyclic aminocarboxylate and aminophosphonic acid ligands. Ligands which form non-ionic (i.e. neutral) metal complexes of gadolinium are known and are described in U.S. Pat. No. 4,885,363. When the radiometal ion is technetium, the chelating group is preferably tetradentate. Preferred chelating groups for technetium are the diaminedioximes, or those having an N₂S₂ or N₃S donor set as described above, of which the N₂S₂ chelating groups are preferred where blood-brain barrier penetration is required.

Further examples of suitable chelating groups in A are disclosed in U.S. Pat. No. 4,647,447, WO89/00557, U.S. Pat. No. 5,367,080, U.S. Pat. No. 5,364,613.

Methods for metallating any chelating group present in the compound of formula (I) are within the level of skill in the art. Metals can be incorporated into a chelating group by any one of three general methods: direct incorporation, template synthesis and/or transmetallation. Direct incorporation is preferred.

Thus it is desirable that the metal ion be easily complexed to the chelating group, for example, by merely exposing or mixing an aqueous solution of the chelating group-containing moiety with a metal salt in an aqueous solution preferably having a pH in the range of about 4 to about 11. The salt can be any salt, but preferably the salt is a water soluble salt of the metal such as a halogen salt, and more preferably such salts are selected so as not to interfere with the binding of the metal ion with the chelating chelating group. The chelating group-containing moiety is preferably in aqueous solution at a pH of between about 5 and about 9, more preferably between pH about 6 to about 8. The chelating group-containing moiety can be mixed with buffer salts such as citrate, carbonate, acetate, phosphate and borate to produce the optimum pH. Preferably, the buffer salts are selected so as not to interfere with the subsequent binding of the metal ion to the chelating group.

As noted above, substrates for ALDH may also be used in radiotherapy, such that the accumulation of radiotherapeutic in the cancer stem cells effectively localises the therapeutic response. Cancer stem cells often show resistance to standard cancer therapeutic methods. Targeted destruction of these cells using an ALDH targeting radiotherapeutic may provide a more effective approach, either on its own or in combination with other cancer therapeutic methods. Cancer therapeutic methods which are conventionally used include chemotherapy, such as with alkylating agents (e.g., cyclophosphamide derivatives including 4-hydroperoxycyclophosphamide, and mafosphamide), hormonal therapy (e.g., with aromatase inhibitors, anti-androgens, or tamoxifen) and radiotherapy.

According to a further aspect of the invention, there is provided a method for radiotherapy of a cancer patient, comprising administration of an effective amount of radiotherapy-labelled substrate for ALDH to said cancer patient.

The “radiotherapy-labelled substrate for ALDH” is a compound of formula (II):

R*-(B)ₘ—C(O)H (II)

or a salt or solvate thereof, wherein
m is an integer 0 or 1;
R* is a radiotherapeutic moiety; and
B is a carrier moiety; and
the compound of formula (II) has a molecular weight of below 800 Daltons.

The term “radiotherapeutic moiety” means a group comprising a therapeutic radionuclide selected from the beta emitters ¹³¹I, ³³P, ¹⁶⁹Er, ¹⁷⁷Lu, ⁶⁷Cu, ¹⁵³Sm, ¹⁹⁸Au, ¹⁰⁹Pd, ¹⁸⁶Re, ¹⁶⁵Dy, ⁸⁹Sr, ³²P, ¹⁸⁸Re, and ⁹⁰Y; alpha emitters ²¹¹At, ²¹²Bi and ²¹³Bi; and Auger emitters ⁵¹Cr, ⁶⁷Ga, ⁷⁵Se, ⁷⁷Br, ¹²³I, ¹¹¹In, ^{99m}Tc and ²⁰¹Tl. When the radiotherapeutic moiety comprises a radioactive metal, the metal is chelated to a chelating group as defined above. The chelating group may be directly bonded to the rest of the compound of formula (II) or may be attached by way of a C₁₋₃₀hydrocarbyl linker group optionally further containing 1 to 10 heteroatoms selected from nitrogen, oxygen, and sulphur which serves to space the chelate sterically from the rest of the compound. Suitable radiotherapeutic moieties comprising a non-metal radiolabel are known in the art, and typically comprise a C₁₋₃₀hydrocarbyl linker group optionally further containing 1 to 10 heteroatoms selected from nitrogen, oxygen, and sulphur and having the non-metal radiolabel covalently attached thereto or incorporated therein or alternatively, in the case of a radiohalo ¹³¹I or ⁷⁷Br, such a radiolabel may be directly bonded to the rest of the compound of formula (II).

In a further aspect of the invention, there is provided a method for detection of tumour stem cells in a subject, comprising:

(i) administration of a compound of formula (Ia), to said subject:

A-(B)ₙ—C(O)H (Ia)

or a salt or solvate thereof, wherein
n is an integer 0 or 1;
A is a radioimaging moiety;
B is a carrier moiety; and
the compound of formula (Ia) has a molecular weight of below 800 Daltons;
(ii) detecting uptake of said compound of formula (Ia) by in vivo radioimaging.

Preferred methods of in vivo radioimaging are PET and SPECT. These imaging methods are well known in the art, and may be used to provide useful information in the management of subjects having or suspected or having a tumour. The properties of the compound of formula (I) or (Ia) allow for selective imaging of ALDH expression during imaging, i.e. identification or quantitative assessment of ALDH expressing cells within a tumour that also contains non-ALDH expressing cells. Analysis of imaging data, for example by comparison of data from ALDH expressing area with adjacent or background areas, will allow estimation of levels of ALDH expression.

The data and images obtained from the imaging methods of the invention may contribute to improved clinical patient management, for example they may provide valuable prognostic information, assist with selection of the most suitable therapy for the subject, or provide a measure of therapy efficacy.

According to a further aspect, the invention provides a method of monitoring the effect of treatment of a tumour in a subject (for example treatment with a cytotoxic agent or radiotherapy), said method comprising:

(i) administration of a compound of formula (I), to said subject:

A-(B)ₙ—C(O)H (I)

or a salt or solvate thereof, wherein
n is an integer 0 or 1;
A is either a radioimaging moiety or an optical imaging moiety;
B is a carrier moiety; and
the compound of formula (I) has a molecular weight of below 800 Daltons;
(ii) detecting uptake of said compound of formula (I) by in vivo imaging, said administration and detection steps (i) and (ii) optionally but preferably being effected repeatedly, for example before, during and after treatment.

In a further aspect of the invention, there is provided a method for detection of tumour stem cells in a subject, comprising:

(i) administration of a compound of formula (Ib), to said subject:

A-(B)ₙ—C(O)H (Ib)

or a salt or solvate thereof, wherein
n is an integer 0 or 1;
A is an optical imaging moiety;
B is a carrier moiety; and
the compound of formula (Ib) has a molecular weight of below 800 Daltons;
(ii) detecting uptake of said compound of formula (Ib) by in vivo optical imaging.

Optical imaging techniques include luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching. Further details of these techniques are provided by: (Tuan Vo-Dinh (editor): “Biomedical Photonics Handbook” (2003), CRC Press LCC; Mycek & Pogue (editors): “Handbook of Biomedical Fluorescence” (2003), Marcel Dekker, Inc.; Splinter & Hopper: “An Introduction to Biomedical Optics” (2007), CRC Press LCC.

The optical imaging methods of the invention may be useful for detecting cancer stem cells in a range of target tissues and conditions, including but not limited to, oesophageal epithelium (squamous or columnar), oesophageal cancer, Barrett's oesophagus, colorectal cancer, skin cancer (for example melanoma), cervical cancer, oral cancer. These imaging methods may provide information that will be useful for the management of patients diagnosed or suspected of having the above conditions. These methods may also be useful during surgery for directing the surgeon and facilitating more accurate identification or removal of cancerous cells.

The compounds of formula (I), (Ia), (Ib), and (II) are substrates for ALDH, having an aldehyde functionality which is converted to a carboxylic acid in vivo, and most preferably selectively by the highly expressed intracellular levels of the enzyme in the cancer stem cell population of the tumour. The negatively charged product of enzyme conversion is trapped within the cell allowing the signal to accumulate over time in vivo.

The optional carrier moiety B is designed to modify the hydrophobicity of the compound of formula (I) or (II) so as to optimize cell permeability, and is suitably of formula:

—(Ar)ₚ—(X¹)_{q}—(C₁₋₆alkyl)ᵣ-

wherein:

p, q, and r are each an integer independently selected from 0 and 1 with the proviso that at least one of p, q, and r is 1;
Ar is a 1, 2, or 3 member aromatic ring system, either fused or unfused, and optionally comprising 1 to 3 heteroatoms selected from nitrogen, oxygen, sulphur, and boron and optionally having from 1 to 5 substituents selected from C₁₋₆alkyl, C₁₋₆haloalkyl, C₁₋₆alkoxy, C₁₋₆haloalkoxy, halo, cyano, nitro, hydroxy, hydroxyC₁₋₆alkyl, and —NR¹R², wherein R¹ and R² are independently selected from hydrogen, C₁₋₆alkyl, and C₁₋₆haloalkyl;
X¹ is selected from —CR²—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, and —CR₂NRCR₂—, wherein each R is independently selected from H, C₁₋₆ alkyl, C₂₋₆ alkenyl, C₂₋₆ alkynyl, C₁₋₆ alkoxyalkyl and C₁₋₆ hydroxyalkyl.

Preferred groups Ar include phenyl, naphthyl, biphenyl, quinoline, isoquinoline, and indole.

In one aspect, the compound of formula (I) as used in the imaging methods of the invention is a compound selected from formulae (Ic) to (Ii):

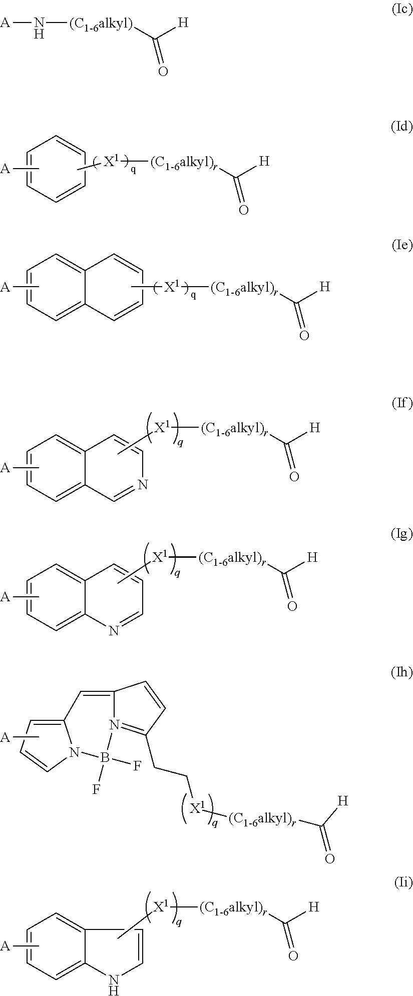

wherein A, X¹, q and r are as defined above and each aryl group optionally has 1 to 5 substituents selected from C₁₋₆alkyl, C₁₋₆haloalkyl, C₁₋₆alkoxy, C₁₋₆haloalkoxy, halo, cyano, nitro, hydroxy, hydroxyC₁₋₆alkyl, and —NR¹R², wherein R¹ and R² are independently selected from hydrogen, C₁₋₆alkyl, and C₁₋₆haloalkyl.

In formulae (Ic) to (Ii), the group A is as defined for formula (I), (Ia), or (Ib) above. In one aspect of the invention, the group A is selected from C₁₋₆radiohaloalkyl such as [¹⁸F]fluoro C₁₋₆alkyl or [^{122, 123, 124}I]iodo C₁₋₆alkyl, C₁₋₆radiohaloalkoxy such as [¹⁸F]fluoro C₁₋₆alkoxy or [^{122, 123, 124}I]iodo C₁₋₆alkoxy, C₁₋₆radiohaloalkylamine such as [¹⁸F]fluoro C₁₋₆alkylNH—, [^{122, 123, 124}I]iodo C₁₋₆alkylNH—, [¹⁸F]fluoro C₁₋₆alkylN(C₁₋₆alkyl)-, [^{122, 123, 124}I]iodo C₁₋₆alkylN(C₁₋₆alkyl)-, [¹⁸F]fluoro, and [^{122, 123, 124}I]iodo.

In formulae (Id) to (Ii), q is an integer 0 or 1 and is preferably 1, and X¹ is as defined above, in one aspect of the invention, X¹ is —CONH— or —SO₂NH—.

In formulae (Id) to (Ii), r is an integer 0 or 1, and is preferably 1.

In one aspect, the compound of formula (Ic) is of formula (Ic*):

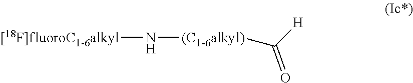

or a salt or solvate thereof.

Particular compounds of formula (Ic*) include:

| Compound No | Structure |
|---|---|
| 1 | 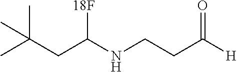 |
| 2 | 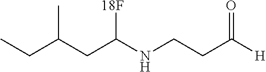 |

In one aspect, the compound of formula (Id) is of formula (Id*)

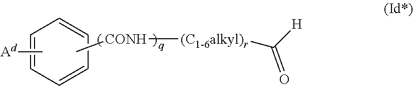

or a salt or solvate thereof wherein:
A^{d} is selected from [¹⁸F]fluoro C₁₋₆alkyl, [^{122, 123, 124}I]iodo C₁₋₆alkyl, [¹⁸F]fluoro C₁₋₆alkoxy, [^{122, 123, 124}I]iodo C₁₋₆alkoxy, [¹⁸F]fluoro C₁₋₆alkylNH—, [^{122, 123, 124}I]iodo C₁₋₆alkylNH—, [¹⁸F]fluoro C₁₋₆alkylN(C₁₋₆alkyl)-, [^{122, 123, 124}I]iodo C₁₋₆alkylN(C₁₋₆alkyl)-, [¹⁸F]fluoro, and [^{122, 123, 124}I]iodo;
q and r are each independently an integer 0 or 1 provided that if r is 0 then q is also 0.

In the compound of formula (Id*), Ad is suitably selected from [¹⁸F]fluoro C₁₋₆alkoxy, [¹⁸F]fluoro, and [^{122, 123, 124}I]iodo, and q is suitably 1.

Particular compounds of formula (Id*) include:

| Compound No | Structure |
|---|---|
| 3 | 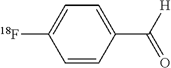 |
| 4 | 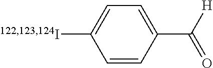 |
| 5 | 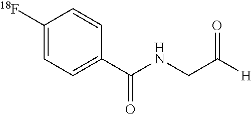 [SMILES: O=CCNC(=O)c1ccc([18F])cc1] |
| 6 | 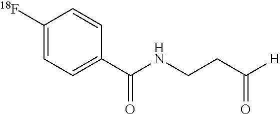 [SMILES: O=CCCNC(=O)c1ccc([18F])cc1] |
| 7 | 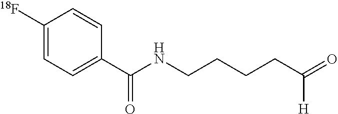 [SMILES: O=CCCCCNC(=O)c1ccc([18F])cc1] |
| 8 | 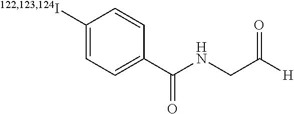 |
| 9 | 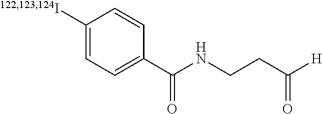 |
| 10 | 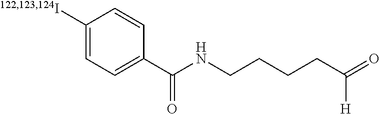 |
| 11 | 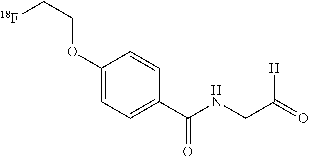 [SMILES: O=CCNC(=O)c1ccc(OCC[18F])cc1] |
| 12 | 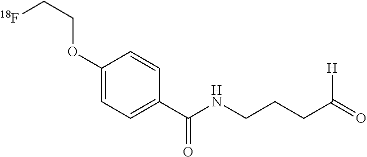 [SMILES: O=CCCCNC(=O)c1ccc(OCC[18F])cc1] |
| 13 | 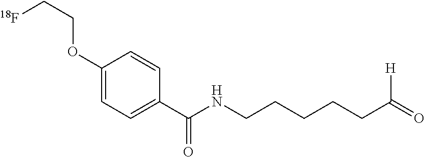 [SMILES: O=CCCCCCNC(=O)c1ccc(OCC[18F])cc1] |
| 14 | 4-[(2-[¹⁸F]fluoroethyl)-propyl-aminolbenzaldehyde; |

In one aspect, the compound of formula (Ie) is of formula (Ie*)

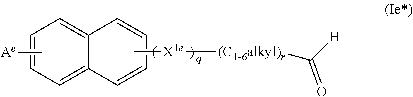

or a salt or solvate thereof wherein:

A^{e} is selected from [¹⁸F]fluoro C₁₋₆alkyl, [^{122, 123, 124}I]iodo C₁₋₆alkyl, [¹⁸F]fluoro C₁₋₆alkoxy, [^{122, 123, 124}I]iodo C₁₋₆alkoxy, [¹⁸F]fluoro C₁₋₆alkylNH—, [^{122, 123, 124}I]iodo C₁₋₆alkylNH—, [¹⁸F]fluoro C₁₋₆alkylN(C₁₋₆alkyl)-, [^{122, 123, 124}I]iodo C₁₋₆alkylN(C₁₋₆alkyl)-, [¹⁸F]fluoro, and [^{122, 123, 124}I]iodo;

### X^{1e} is —CONH— or —SO₂NH—;

q and r are each independently an integer 0 or 1 provided that if r is 0 then q is also 0;
and the naphthyl ring is optionally further substituted with 1 to 3 substituents selected from C₁₋₆alkyl, C₁₋₆haloalkyl, C₁₋₆alkoxy, C₁₋₆haloalkoxy, halo, cyano, nitro, hydroxy, hydroxyC₁₋₆alkyl, and —NR¹R², wherein R¹ and R² are independently selected from hydrogen, C₁₋₆alkyl, and C₁₋₆haloalkyl.

In the compound of formula (Ie*), A^{e} is preferably selected from [¹⁸F]fluoro, and [^{122, 123, 124}I]iodo, and the naphthyl ring is suitable substituted by a group —NR¹—R², wherein R¹ and R² are independently selected from hydrogen, C₁₋₆alkyl, and C₁₋₆haloalkyl.

Particular compounds of formula (Ie*) include:

| Compound | |
|---|---|
| No | Structure |
| 15 | 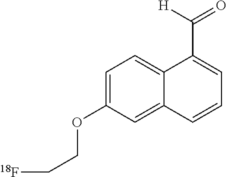 [SMILES: O=Cc1cccc2cc(OCC[18F])ccc12] |
| 15a | 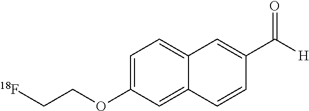 [SMILES: O=Cc1ccc2cc(OCC[18F])ccc2c1] |
| 16 | 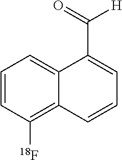 [SMILES: O=Cc1cccc2c([18F])cccc12] |
| 17 | 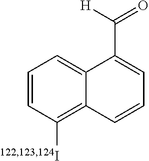 |
| 18 | 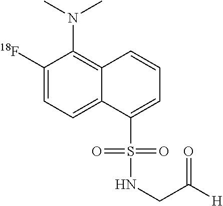 [SMILES: CN(C)c1c([18F])ccc2c(S(=O)(=O)NCC=O)cccc12] |
| 19 | 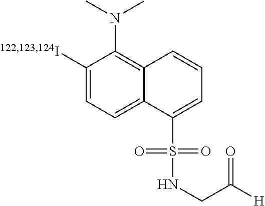 |
| 20 | 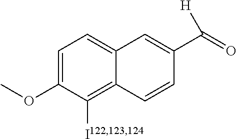 |

In one aspect, the compound of formula (If) is of formula (If*)

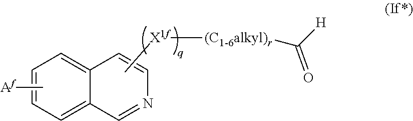

or a salt or solvate thereof wherein:

N is selected from [¹⁸F]fluoro C₁₋₆alkyl, [^{122, 123, 124}I]iodo C₁₋₆alkyl, [¹⁸F]fluoro C₁₋₆alkoxy, [^{122, 123, 124}I]iodo C₁₋₆alkoxy, [¹⁸F]fluoro C₁₋₆alkylNH—, [^{122, 123, 124}I]iodo C₁₋₆alkylNH—, [¹⁸F]fluoro C₁₋₆alkylN(C₁₋₆alkyl)-, [^{122, 123, 124}I]iodo C₁₋₆alkylN(C₁₋₆alkyl)-, [¹⁸F]fluoro, and [^{122, 123, 124}I]iodo;

### X^{1f} is —CONH— or —SO₂NH—;

q and r are each independently an integer 0 or 1 provided that if r is 0 then q is also 0; and the isoquinoline ring is optionally further substituted with 1 to 3 substituents selected from C₁₋₆alkyl, C₁₋₆haloalkyl, C₁₋₆alkoxy, C₁₋₆haloalkoxy, halo, cyano, nitro, hydroxy, hydroxyC₁₋₆alkyl, and —NR¹—R², wherein R¹ and R² are independently selected from hydrogen, C₁₋₆alkyl, and C₁₋₆haloalkyl.

Particular compounds of formula (If*) include:

| Compound No | Structure |
|---|---|
| 21 | 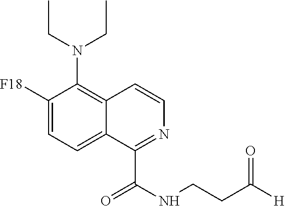 |
| 22 | 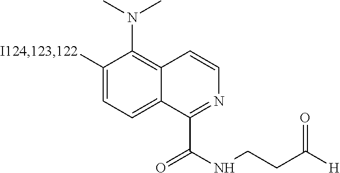 |
| 23 | 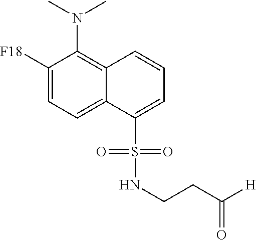 |
| 24 | 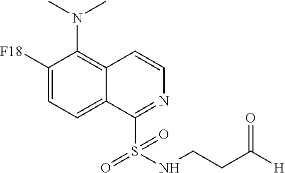 |

In one aspect, the compound of formula (Ig) is of formula (Ig*)

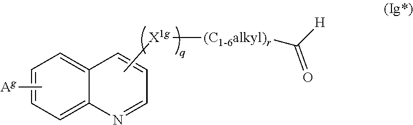

or a salt or solvate thereof wherein:
A^{g} is selected from [¹⁸F]fluoro C₁₋₆alkyl, [^{122, 123, 124}I]iodo C₁₋₆alkyl, [¹⁸F]fluoro C₁₋₆alkoxy, [^{122, 123, 124}I]iodo C₁₋₆alkoxy, [¹⁸F]fluoro C₁₋₆alkylNH—, [^{122, 123, 124}I]iodo C₁₋₆alkylNH—, [¹⁸F]fluoro C₁₋₆alkylN(C₁₋₆alkyl)-, [^{122, 123, 124}I]iodo C₁₋₆alkylN(C₁₋₆alkyl)-, [¹⁸F]fluoro, and [^{122, 123, 124}I]iodo;

### X^{1g} is —CONH— or —SO₂NH—;

q and r are each independently an integer 0 or 1 provided that if r is 0 then q is also 0; and the quinoline ring is optionally further substituted with 1 to 3 substituents selected from C₁₋₆alkyl, C₁₋₆haloalkyl, C₁₋₆alkoxy, C₁₋₆haloalkoxy, halo, cyano, nitro, hydroxy, hydroxyC₁₋₆alkyl, and —NR¹R², wherein R¹ and R² are independently selected from hydrogen, C₁₋₆alkyl, and C₁₋₆haloalkyl.

Particular compounds of formula (Ig*) include:

| Compound No | Structure |
|---|---|
| 25 | 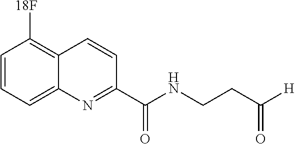 |
| 26 | 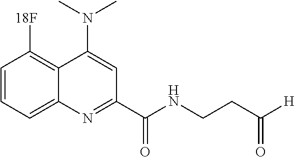 |
| 27 | 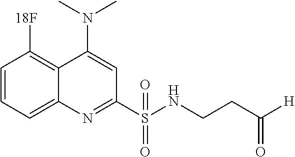 |
| 28 | 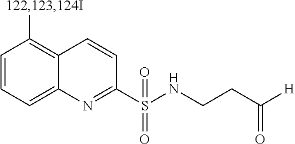 |

In one aspect, the compound of formula (Ih) is of formula (Ih*):

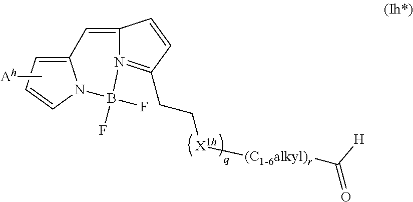

or a salt or solvate thereof wherein:
A^{h} is absent or is selected from [¹⁸F]fluoro C₁₋₆alkyl, [^{122, 123, 124}I]iodo C₁₋₆alkyl, [¹⁸F]fluoro C₁₋₆alkoxy, [^{122, 123, 124}I]iodo C₁₋₆alkoxy, [¹⁸F]fluoro C₁₋₆alkylNH—, [^{122, 123, 124}I]iodo C₁₋₆alkylNH—, [¹⁸F]fluoro C₁₋₆alkylN(C₁₋₆alkyl)-, [^{122, 123, 124}I]iodo C₁₋₆alkylN(C₁₋₆alkyl)-, [¹⁸F]fluoro, and [^{122, 123, 124}I]iodo;

### X^{1h} is —CONH— or —SO₂NH—;

q and r are each independently an integer 0 or 1 provided that if r is 0 then q is also 0; and the aromatic ring is optionally further substituted with 1 to 3 substituents selected from C₁₋₆alkyl, C₁₋₆haloalkyl, C₁₋₆alkoxy, C₁₋₆haloalkoxy, halo, cyano, nitro, hydroxy, hydroxyC₁₋₆alkyl, and —NR¹R², wherein R¹ and R² are independently selected from hydrogen, C₁₋₆alkyl, and C₁₋₆haloalkyl.

Compounds of formula (Ih*) in which the group A^{h} is absent form a separate aspect of the invention, in which the aryl ring is the optical imaging moiety.

Particular compounds of formula (Ih*) include:

| Compound No | Structure |
|---|---|
| 29 | 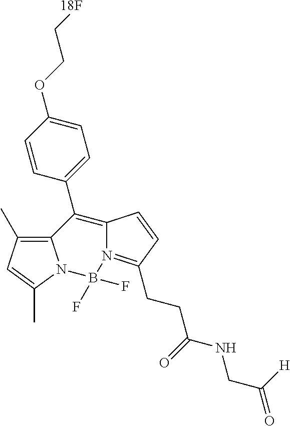 |
| 30 | 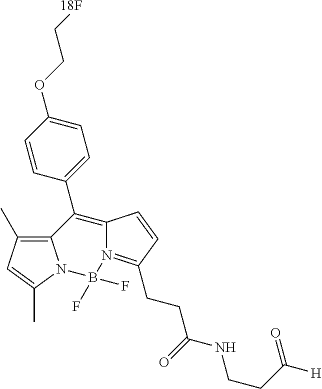 |

In one aspect, the compound of formula (Ii) is of formula (Ii*):

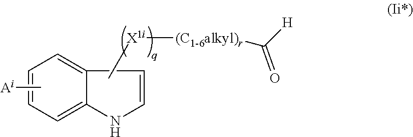

or a salt or solvate thereof wherein:
Aⁱ is selected from [¹⁸F]fluoro C₁₋₆alkyl, [^{122, 123, 124}I]iodo C₁₋₆alkyl, [¹⁸F]fluoro C₁₋₆alkoxy, [^{122, 123, 124}I]iodo C₁₋₆alkoxy, [¹⁸F]fluoro C₁₋₆alkylNH—, [^{122, 123, 124}I]iodo C₁₋₆alkylNH—, [¹⁸F]fluoro C₁₋₆alkylN(C₁₋₆alkyl)-, [^{122, 123, 124}I]iodo C₁₋₆alkylN(C₁₋₆alkyl)-, [¹⁸F]fluoro, and [^{122, 123, 124}I]iodo;

### X¹ⁱ is —CONH— or —SO₂NH—;

q and r are each independently an integer 0 or 1 provided that if r is 0 then q is also 0; and the indole ring is optionally further substituted with 1 to 3 substituents selected from C₁₋₆alkyl, C₁₋₆haloalkyl, C₁₋₆alkoxy, C₁₋₆haloalkoxy, halo, cyano, nitro, hydroxy, hydroxyC₁₋₆alkyl, and —NR¹R², wherein R¹ and R² are independently selected from hydrogen, C₁₋₆alkyl, and C₁₋₆haloalkyl.

Particular compounds of formula (Ii*) include:

| Compound No | Structure |
|---|---|
| 31 | 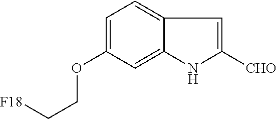 |
| 32 | 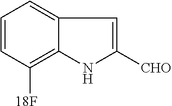 |
| 33 | 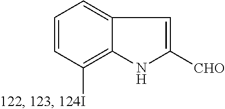 |
| 34 | 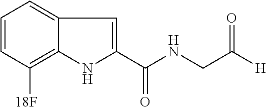 |
| 35 | 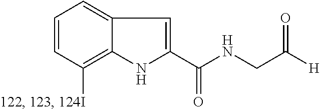 |

In one aspect, the compound of formula (II) as used in the radiotherapy methods of the invention is a compound selected from formulae (IIc) to (IIi):

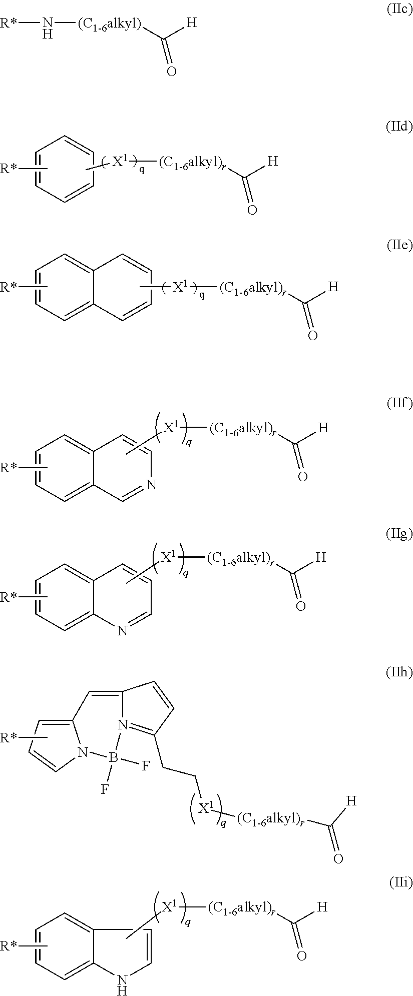

wherein R*, X¹, q and r are as defined above and each aryl group optionally has 1 to 5 substituents selected from C₁₋₆alkyl, C₁₋₆haloalkyl, C₁₋₆alkoxy, C₁₋₆haloalkoxy, halo, cyano, nitro, hydroxy, hydroxyC₁₋₆alkyl, and —NR¹R², wherein R¹ and R² are independently selected from hydrogen, C₁₋₆alkyl, and C₁₋₆haloalkyl.

Certain compounds of formula (Ic) to (Ii), (Ic*) to (Ii*), and (IIc) to (IIi) are novel and therefore form a further aspect of the invention.

The compounds of formula (I) and (II) as well as the more specific aspects thereof, may be prepared by conventional techniques, for example as described below and in the examples. Incorporation of the radioimaging moiety or optical imaging moiety into a compound of formula (I) or of a radiotherapeutic moiety into a compound of formula (II) is suitably effected as close to the end of synthesis as possible, so as to avoid unnecessary decay or loss of thereof.

A ¹¹C label may be incorporated into a compound of the invention by way of a ¹¹C-labelling agent, i.e. a small reactive molecule capable of reacting with a functional group in a precursor to the compound of the invention. Examples of such labelling agents include [¹¹C]carbon dioxide, [¹¹C]carbon monoxide, [¹¹C]methane, [¹¹C]methyl iodide, [¹¹C]phosgene, [¹¹C]cyanide, [¹¹C]cyanamide, and [¹¹C]guanidine. Of these, the most commonly used are [¹¹C]carbon dioxide and [¹¹C]methyl iodide. A thorough review of such ¹¹C-labelling techniques may be found in Antoni et al “Aspects on the Synthesis of ¹¹C-Labelled Compounds” in Handbook of Radiopharmaceuticals, Ed. M. J. Welch and C. S. Redvanly (2003, John Wiley and Sons).

¹¹C is produced as ¹¹CO₂ or ¹¹CH₄, from N₂ target gas with a trace of O₂ or H₂ respectively, via the ¹⁴N(p,α)¹¹C nuclear reaction (Bida et al, Radiochim. Acta., 27 91979) 181). Either of ¹¹CO₂ or ¹¹CH₄ may be converted to useful ¹¹C-labelling agents such as [¹¹C]methyl iodide.

[¹¹C]methyl iodide is commonly used to effect [¹¹C]methylation of a carbon, nitrogen, oxygen, or sulphur nucleophile, for example an amine or hydroxy group. The reactivity of the electrophilic carbon in [¹¹C]methyl iodide may be increased by conversion to, for example, [¹¹C]methyl triflate (Holschbach and Schuller, Appl. Radiat. Isot., 44 (1993), 897). Alternatively, [¹¹C]methyl iodide may be converted to nucleophilic [¹¹C]methyl lithium or a lithium [¹¹C]methyl(2-thienyl)cuprate which broadens the spectrum of functionalities which can be labelled by [¹¹C]methylation. [¹¹C]methyl iodide may also be converted to further labelling agents such as [¹¹C]methylhypofluorite, triphenylarsonium [¹¹C]methylide, or [¹¹C]methylmagnesium iodide. [¹¹C]methylation may be carried out in solution phase, dissolving the appropriate precursor in a solvent such as dimethylsulphoxide, dimethylformamide, acetonitrile, or acetone, and in the presence of a base, for example potassium carbonate, sodium hydroxide, or sodium hydride. Alternatively, [¹¹C]methylation may be performed using a solid support such as an HPLC loop or a solid phase extraction cartridge to first immobilise the precursor before passing through the [¹¹C]methylation agent.

Higher [¹¹C]alkyl halides, such as [¹¹C]ethyliodide or benzyl halides may be prepared from [¹¹C]carbon dioxide by reaction with a Grignard reagent followed by reduction with lithium aluminium hydride and halogenation, for example, iodination with hydroiodic acid. These halides are used in a similar way to [¹¹C]methyl iodide for alkylation of a carbon, nitrogen, oxygen, or sulphur nucleophile.

[¹¹C]acyl chlorides such as acetyl chloride, cyclohexanecarbonyl chloride and furoyl chloride may be used for labelling of carbonyl positions, as described for example in McCarron et al, J. Labelled Compd. Radiopharm, 38, 941-953. Carbonyl positions may also be labelled using [¹¹C]phosgene or [¹¹C]carbon monoxide.

[¹¹C] cyanogen bromide may be used for unspecific labelling of macromolecules and for chemoselective labelling of cyanamides, cyanates, and thiocyanates by reaction with amines, alcohols, and thiols respectively.

Incorporation of a [¹¹C]label in an aromatic ring may be achieved by the methods of Mäding et al (2000) J. Labelled Compd. Radiopharm. 39, 585-600, and in a heterocyclic ring by the methods of Thorell et al (1998), J. Labelled Compd. Radiopharm. 41, 345-353.

¹⁸F may be incorporated into a compound of the invention either by nucleophilic or electrophilic fluorination methods. The fluorine may be incorporated directly, for example, by nucleophilic displacement of a leaving group by [¹⁸F]fluoride, or by way of a ¹⁸F-fluorinated labelling agent which is prepared and then attached to the target molecule by a second reaction, such as an alkylation.

[¹⁸F]fluoride is conveniently prepared from ¹⁸O-enriched water using the (p,n)-nuclear reaction, (Guillaume et al, Appl. Radiat. Isot. 42 (1991) 749-762) and generally isolated as the potassium salt which is dried and solubilised with a phase transfer agent such as a tetraalkylammonium salt or an aminopolyether (for example, Kryptofix 2.2.2). Nucleophilic displacement of a leaving group, often a sulphonate ester, such as a p-toluenesulphonate, trifluoromethanesulphonate, or methanesulphonate, nitro, triC₁₋₄alkylammonium group, or a halo group such as iodo or bromo, may typically be effected by heating for 10 to 30 minutes at elevated temperatures, for example 80 to 160° C., suitably 60 to 120° C., or by microwave heating, in a polar aprotic solvent such as acetonitrile, dimethylsulphoxide, or dimethylformamide.

Useful [¹⁸F]labelling agents include the [¹⁸F]fluoroalkylhalides, such as [¹⁸F]fluoropropylbromide. These are routinely prepared by nucleophilic displacement of a suitable leaving group by [¹⁸F]fluoride before being coupled to a suitable precursor.

Electrophilic [¹⁸F]fluorination may be performed using ¹⁸F₂, alternatively the ¹⁸F₂ may be converted to [¹⁸F]acetylhypofluorite (Lerman et al, Appl. Radiat. Isot. 49 (1984), 806-813) or to a N-[18F]fluoropyridinium salt (Oberdorfer et al, Appl. Radiat. Isot. 39 (1988), 806-813). These electrophilic reagents may be used to incorporate ¹⁸F by performing double bond addition, aromatic substitution reactions, for example substitution of a trialkyl tin or mercury group, or fluorination of carbanions.

⁷⁶Br is usually produced by the reaction ⁷⁶Se[p,n]⁷⁶Br (Friedman et al, J Label Compd Radiopharm, 1982, 19, 1427-8) and used as a bromide salt such as ammonium bromide or sodium bromide. ¹²⁴I is commonly obtained by the reaction ¹²⁴Te (p,n)¹²⁴I and used as an iodide salt such as sodium iodide. Other isotopes of bromine and iodine may be prepared by analogy. Radiobromo and radioiodo are commonly introduced to an organic molecule by electrophilic bromination or iodination of a trialkyltin precursor, such as a tributylstannyl compound, in the presence of an oxidising agent such as peracetic acid, N-chlorosuccinimide, and N-chlorotolylsulphonamide (for example chloramine-T or Iodogen) or by indirect methods such as use of Bolton Hunter reagent at non-extreme temperature and in a suitable solvent such as an aqueous buffer. Radiohalogenation methods are reviewed in detail in Bolton, J. Label. Compd Radiopharm 2002, 45, 485-528.

Radiometals may be incorporated into a chelating group as described above.

An optical imaging moiety may be conjugated with an appropriate precursor to form a compound of the invention by conventional methods—for example, see Achilefu, Technol. Cancer. Res. Treat., 3, 393-409 (2004); Li et al Org. Lett., 8(17), 3623-26 (2006); and Bullok et al, J. Med. Chem., 48, 5404-5407 (2005). General methods for conjugation of cyanine dyes are described by Licha et al Topics Curr. Chem., 222, 1-29 (2002); Adv. Drug Deliv. Rev., 57, 1087-1108 (2005). For reviews and examples of labelling using fluorescent dye labelling reagents, see “Non-Radioactive Labelling, a Practical Introduction”, Garman, A. J. Academic Press, 1997; “Bioconjugation—Protein Coupling Techniques for the Biomedical Sciences”, Aslam, M. and Dent, A., Macmillan Reference Ltd, (1998).

Reagents suitable for incorporating an optical imaging moiety into a compound of the invention are commercially available from GE Healthcare Limited, Atto-Tec, Dyomics, Molecular Probes and others. Most such dyes are available as NHS (N-hydroxy succinimide) activated esters.

During incorporation of the radioimaging moiety or optical imaging moiety into a compound of formula (I) or of a radiotherapeutic moiety into a compound of formula (II) the aldehyde function is optionally blocked as a protecting group to avoid unwanted side-reaction. Suitable protecting groups for this purpose include an acetal such as —CH(—O—C₁₋₄alkyl-O—) (for example —CH(—OCH₂CH₂O—); or —CH(OC₁₋₄alkyl)₂ (for example —CH(OCH₃)₂). Subsequent deprotection to form the free aldehyde may be effected using standard methods such as treatment with acid. In one embodiment the aldehyde is present in the free form with no protection during incorporation of the radioimaging moiety or optical imaging moiety into a compound of formula (I) or of a radiotherapeutic moiety into a compound of formula (II).

Compounds of formula (Ic*) may be prepared according to scheme 1, or by methods analogous thereto. Further details of analogous chemistry may be found in WO1996/036344; Zhurnal Obshchei Khimii; 19; 1949, 110; Chem. Abstr. 1949; 6164; and WO2004/9528 A1. The starting amine is commercially available.

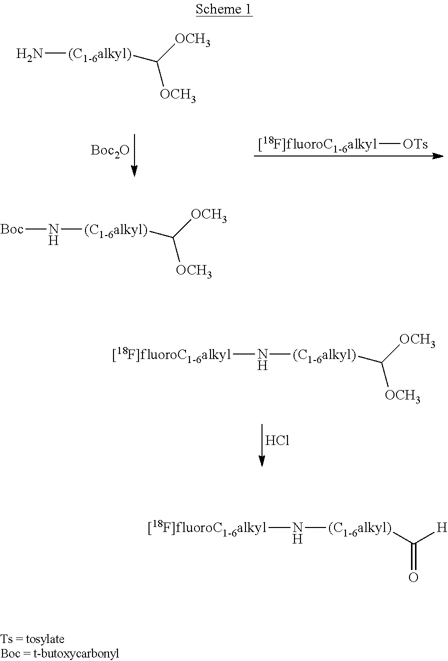

Compounds of formula (Id*) may be prepared according to scheme 2 or 3, or by methods analogous thereto.

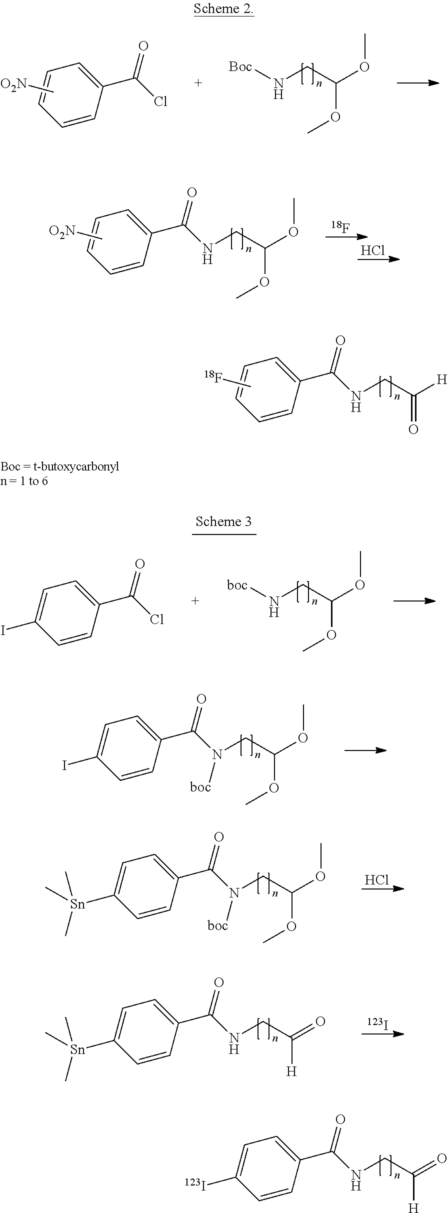

Compounds of formula (Ie*) may be prepared according to Scheme 4 to 7, or by methods analogous thereto. Further details of analogous chemistry may be found in WO 2005/021553 A1; Tetrahedron Letters 44 (2003) 2691-2693; and WO1996/036344.

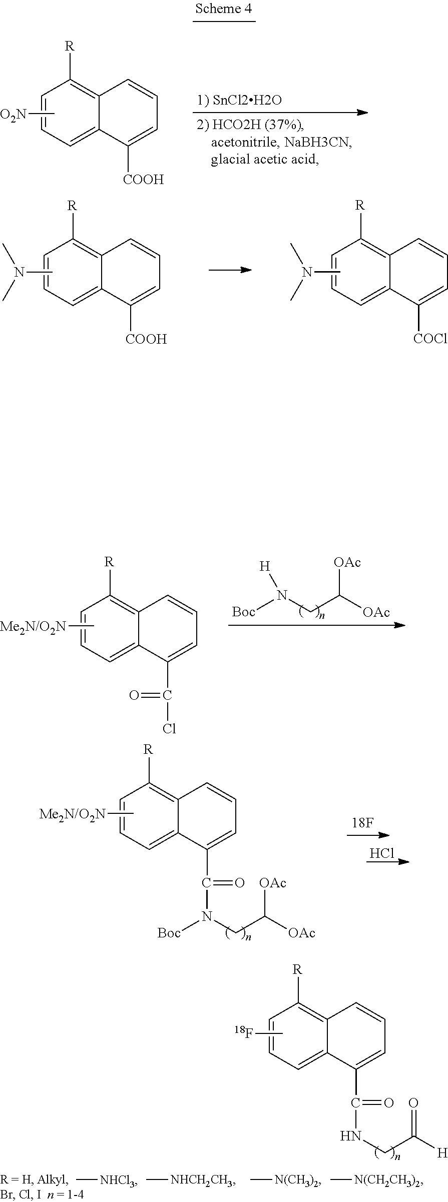

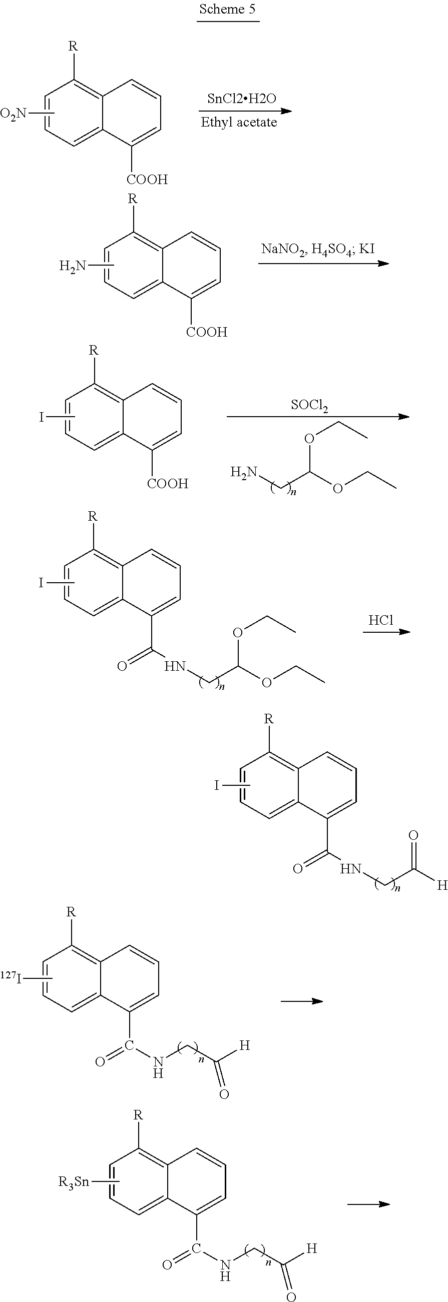

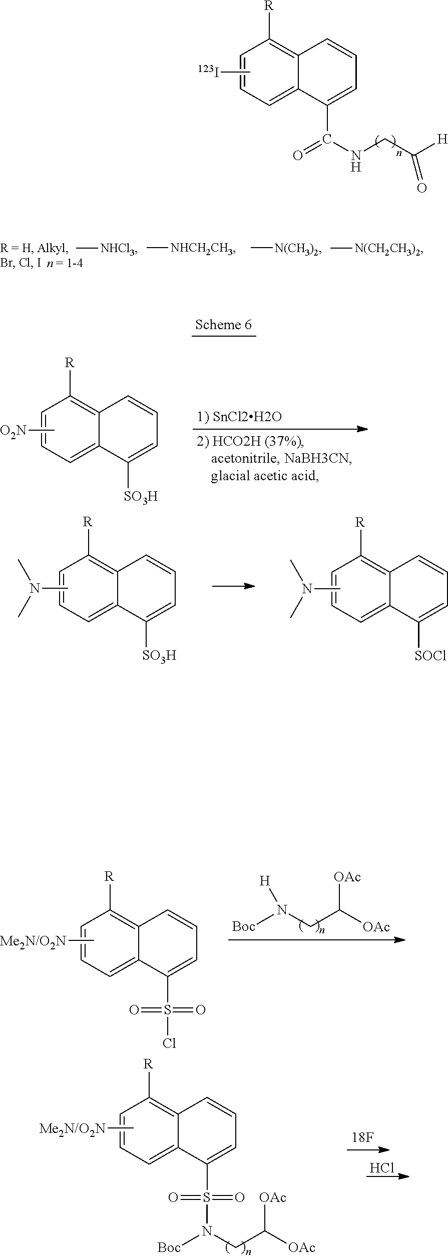

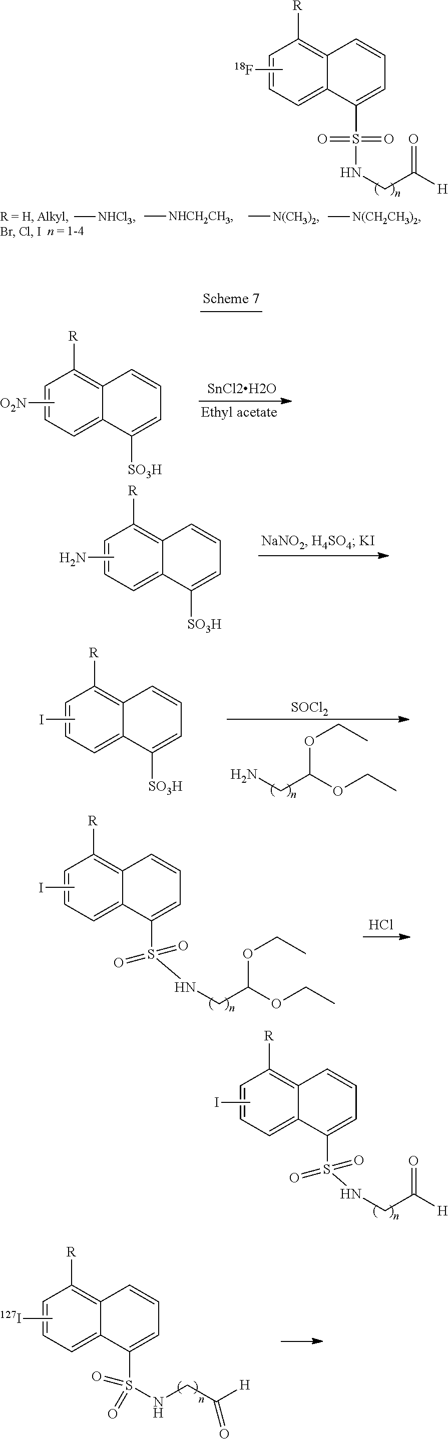

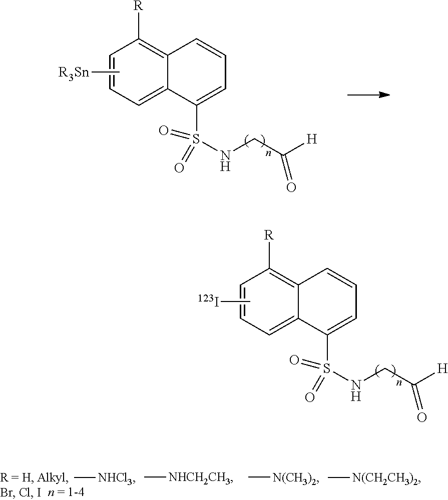

Compounds of formula (If*) may be prepared according to scheme 8 or 9, or by methods analogous thereto. Further details of analogous chemistry may be found in JOC, December, 4571-79, 1962; Tetrahedron Letters 44 (2003) 2691-2693; and WO1996/036344.

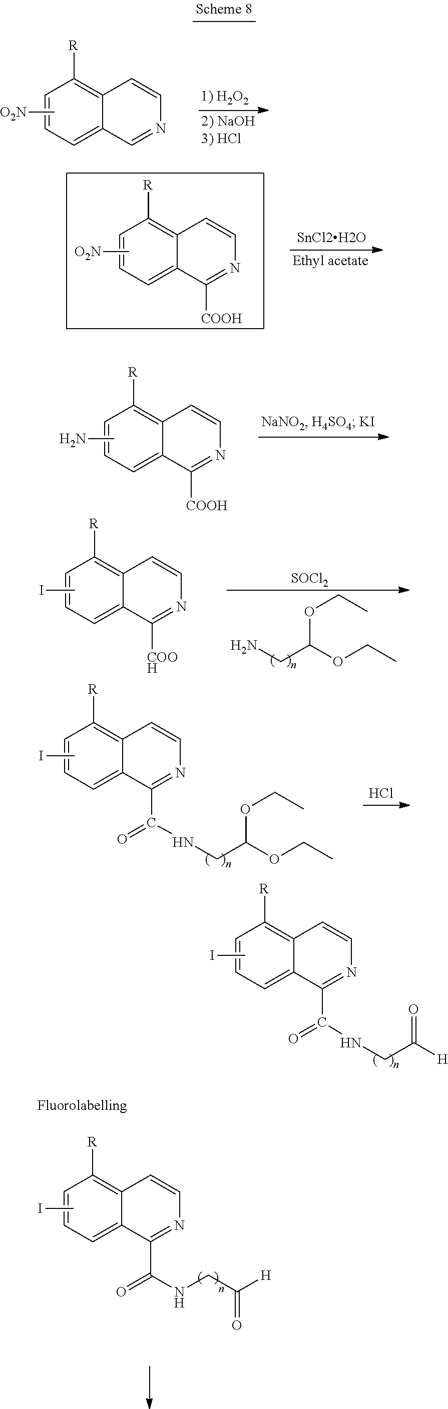

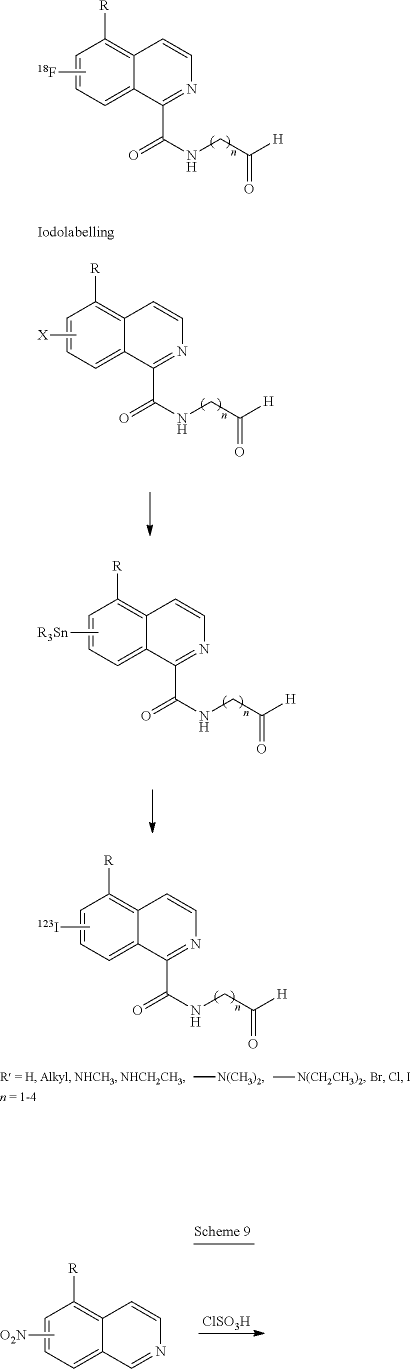

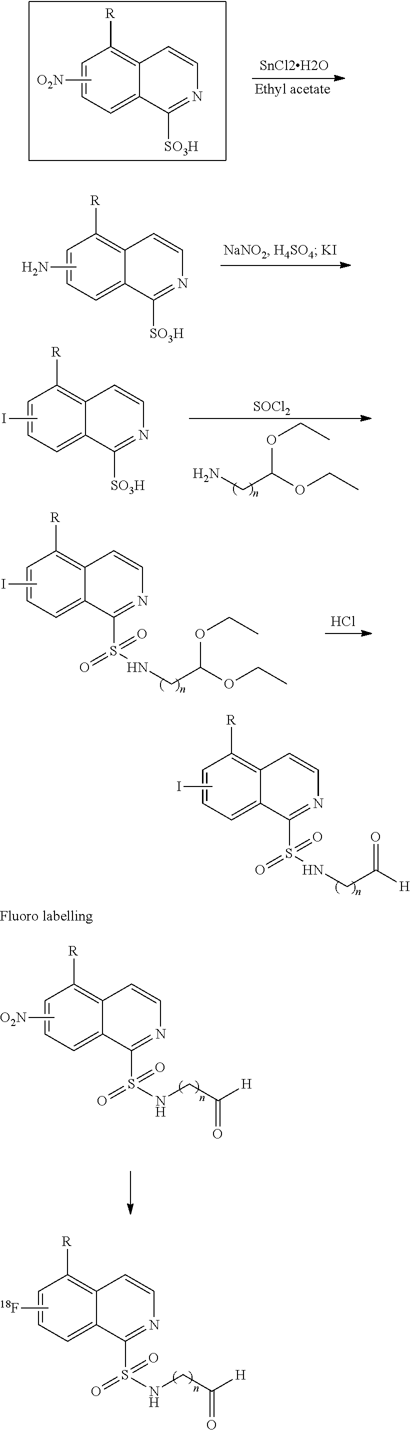

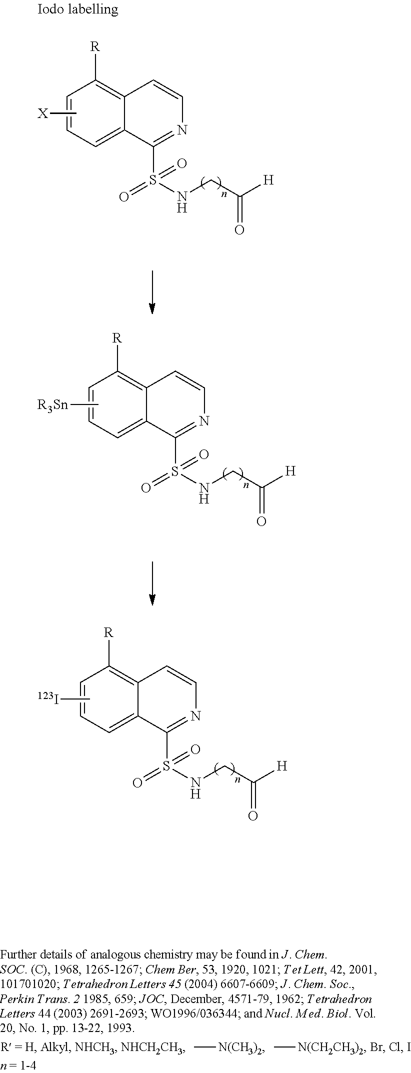

Compounds of formula (If*) may be prepared according to scheme 10 to 12, or by methods analogous thereto. The starting materials may be obtained by analogy to the chemistry described above, from the corresponding nitro-quinoline-2-carboxylic acid which is commercially available. Further details of analogous chemistry may be found in Tetrahedron Letters 44 (2003) 2691-2693; WO1996036344; Nucl. Med. Biol. Vol. 20, No. I, pp. 13-22, 1993

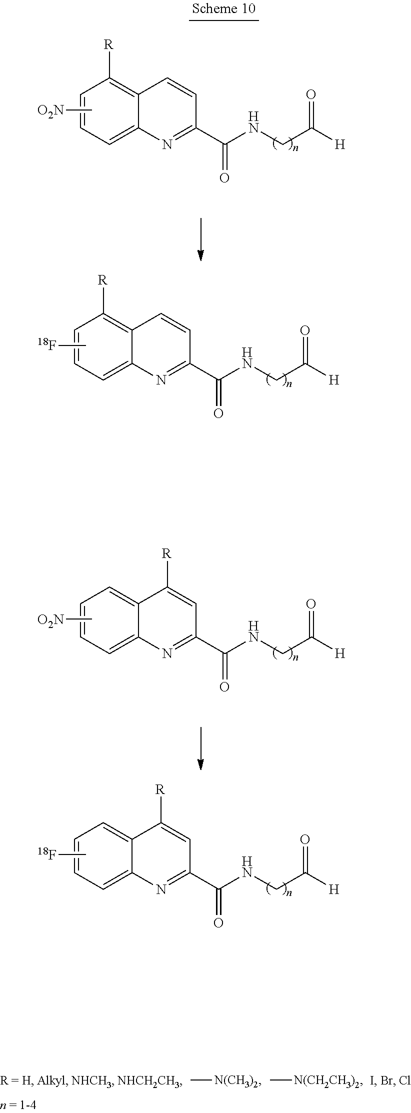

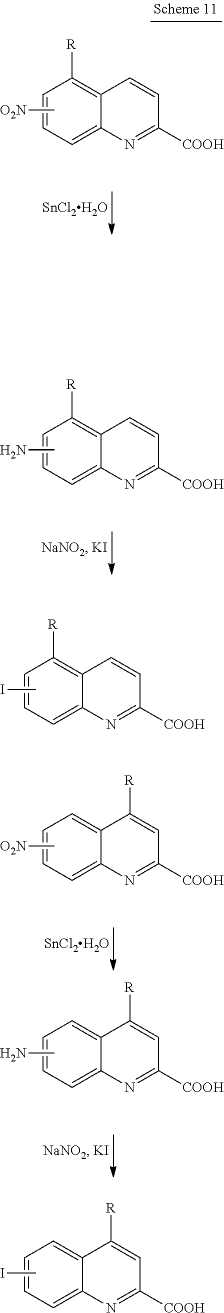

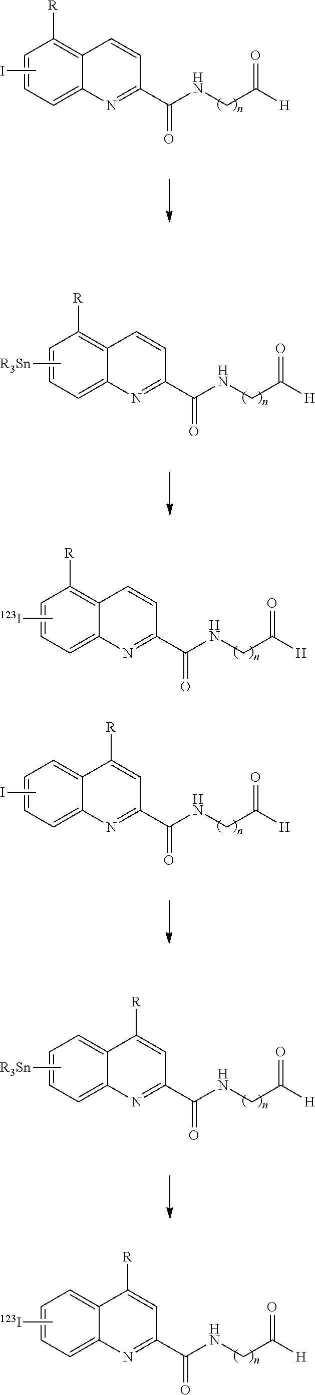

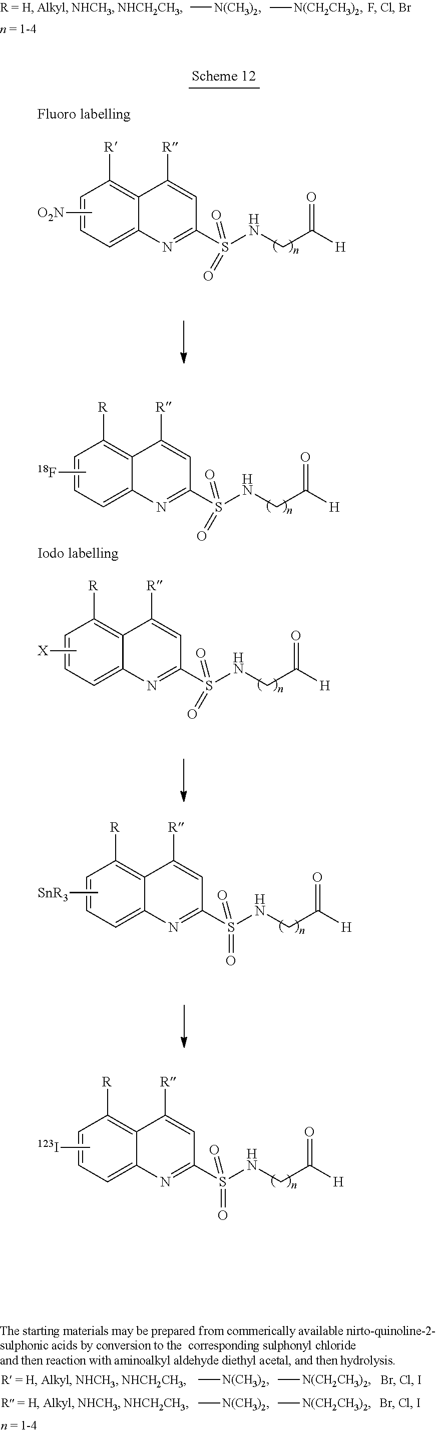

A compound of formula (I), (Ia) to (Ii), (Ic*) to (Ii*), (II), (IIc) to (IIi), or a salt or solvate thereof is preferably administered for in vivo use in a pharmaceutical formulation comprising the compound of the invention and a pharmaceutically acceptable excipient, such formulations thus form a further aspect of the invention. A “pharmaceutical formulation” is defined in the present invention as a formulation comprising an effective amount of a compound of formula (I), (Ia) to (Ii), (Ic*) to (Ii*), (II), (IIc) to (IIi), or a salt or solvate thereof in a form suitable for administration to a mammal, suitably a human. The “pharmaceutically acceptable excipient” is a fluid, especially a liquid, in which the compound of the invention can be suspended or dissolved, such that the formulation is physiologically tolerable, ie. can be administered to the mammalian body without toxicity or undue discomfort. The pharmaceutically acceptable excipient is suitably an injectable carrier liquid such as sterile, pyrogen-free water for injection; an aqueous solution such as saline (which may advantageously be balanced so that the final formulation for injection is isotonic); an aqueous solution of one or more tonicity-adjusting substances (for example, salts of plasma cations with biocompatible counterions), sugars (for example, glucose or sucrose), sugar alcohols (for example, sorbitol or mannitol), glycols (for example. glycerol), or other non-ionic polyol materials (for example, polyethyleneglycols, propylene glycols and the like). Preferably the pharmaceutically acceptable excipient is pyrogen-free water for injection or isotonic saline.

The pharmaceutical formulation may optionally contain additional excipients such as an antimicrobial preservative, pH-adjusting agent, filler, stabiliser or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical formulation. The antimicrobial preservative may, however, also optionally be used to inhibit the growth of potentially harmful micro-organisms in one or more components of kits used to prepare said pharmaceutical formulation prior to administration. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the pharmaceutical formulation is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the pharmaceutical formulation is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

Administration for radioimaging or radiotherapy methods is preferably carried out by injection of the pharmaceutical formulation as an aqueous solution. Such a formulation may optionally contain further excipients as described above, more typically including one or more excipient such as buffers; pharmaceutically acceptable solubilisers (e.g. cyclodextrins or surfactants such as Pluronic, Tween or phospholipids); pharmaceutically acceptable stabilisers or antioxidants (such as ascorbic acid, gentisic acid or para-aminobenzoic acid). For optical imaging methods, administration of the pharmaceutical formulation of the invention may be topical.

The pharmaceutical formulations of the invention are typically supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical formulations of the present invention preferably have a dosage suitable for a single patient and are provided in a suitable syringe or container, as described above.

The pharmaceutical formulations of the invention may be prepared under aseptic manufacture (ie. clean room) conditions to give the desired sterile, non-pyrogenic product. It is preferred that the key components, especially the excipients plus those parts of the apparatus which come into contact with the pharmaceutical formulation (for example, vials) are sterile. The components of the pharmaceutical formulation can be sterilised by methods known in the art, including: sterile filtration, terminal sterilisation using, for example, gamma-irradiation, autoclaving, dry heat or chemical treatment (for example, with ethylene oxide). It is preferred to sterilise some components in advance, so that the minimum number of manipulations needs to be carried out. As a precaution, however, it is preferred to include at least a sterile filtration step as the final step in the preparation of the pharmaceutical formulation.

An “effective amount” of a compound of formula (I), (Ia) to (Ii), (Ic*) to (Ii*) or (II), (IIc) to (IIi) or a salt or solvate thereof means an amount which is effective for use in in vivo imaging (PET, SPECT, or Optical) or for use in radiotherapy and will vary depending on the exact compound to be administered, the weight of the subject or patient, and other variables as would be apparent to a physician skilled in the art. The radiolabelled compounds of this invention may be administered to a subject for PET or SPECT imaging in amounts sufficient to yield the desired signal, typical radionuclide dosages of 0.01 to 100 mCi, preferably 0.1 to 50 mCi will normally be sufficient per 70 kg bodyweight. Likewise for radiotherapy an acceptable dose not exceeding the maximum tolerated dose for the bone marrow (typically 200-300 cGy) is employed.

In a further aspect of the invention, there is provided a compound of formula (I), (Ia) to (Ii), (Ic*) to (Ii*) or (II), (IIc) to (IIi) or a salt or solvate of any thereof, for use in medicine.

### EXAMPLES

The invention is illustrated by way of examples in which the following abbreviations are used:

DMF: N,N′-dimethylformamide;
TFA: trifluoroacetic acid;
min(s): minute(s);
HPLC: high performance liquid chromatography;
THF: tetrahydrofuran;
NMR: nuclear magnetic resonance

### Example 1

### Preparation of 2-[2-(2-fluoromethyl-phenylsulfanyl)-ethyl]-aldehyde

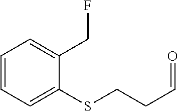 [SMILES: O=CCCSc1ccccc1CF]

### 1a) Synthesis of [2-(2-[1,3]dioxolan-2-ylethylsulfanyl)phenyl]methanol

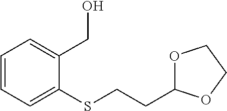 [SMILES: OCc1ccccc1SCCC1OCCO1]

2-(2-Bromoethyl)-1,3-dioxolane (223 μl, 1.86 mmol) was added to 2-mercaptobenzyl alcohol (52.3 mg, 0.37 mmol) and potassium carbonate (102.3, 0.74 mmol) in DMF. The mixture was stirred at room temperature over night before DMF was evaporated under reduced pressure and the crude product purified by reverse phase preparative chromatography (Vydac 218TP1022 column; solvents A=water/0.1% TFA and B=CH₃CN/0.1% TFA; gradient 10-50% B over 40 min; flow 10 ml/min; detection at 214 nm). A yield of 65.1 mg of purified material was obtained (Analytical HPLC: Vydac 218TP54 column; solvents: A=water/0.1% TFA and B=CH₃CN/0.1% TFA; gradient 10-50% B over 20 min; flow 1.0 ml/minute; retention time 15.017 minutes detected at 214 and 254 nm).

### 1b) Synthesis of 2-[2-(2-chloromethyl-phenylsulfanyl)-ethyl]-[1,3]dioxolane

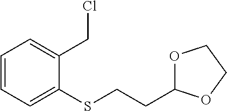 [SMILES: ClCc1ccccc1SCCC1OCCO1]

Mesyl chloride (65 μl, 0.83 mmol) was added to a solution of [2-(2-[(1,3]dioxolan-2-yl-ethylsulfanyl)-phenyl]-methanol (40 mg, 0.17 mmol) and triethyl amine (116 μl, 0.83 mmol) in THF. After 5 days the precipitate was filtered of and THF evaporated under reduced pressure and the crude product purified by reverse phase preparative chromatography (Vydac 218TP1022 column; solvents A=water/0.1% TFA and B=CH₃CN/0.1% TFA; gradient 40-80% B over 40 min; flow 10 ml/minute; detection at 254 nm). The fractions were left in the fridge overnight and to the acetonitrile phase was added diethyl ether, dried (Na₂SO₄) and evaporated under reduced pressure. A yield of 24.5 mg of purified material was obtained (Analytical HPLC: Vydac 218TP54 column; solvents: A=water/0.1% TFA and B=CH₃CN/0.1% TFA; gradient 40-80% B over 20 min; flow 1.0 ml/minute; retention time 10.4 minutes detected at 214 and 254 nm). Structure verified by NMR.

### 1c) Synthesis of 2-[2-(2-fluoromethyl-phenylsulfanyl)-ethyl]-[1,3]dioxolane

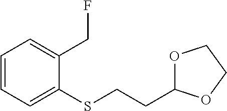 [SMILES: FCc1ccccc1SCCC1OCCO1]

Potassium fluoride (3.5 mg, 0.060 mmol) and kryptofix 222 (22.5 mg, 0.060 mmol) were dissolved in acetonitrile (1 ml) and added to 2-[2-(2-chloromethyl-phenylsulfanyl)-ethyl]-[1,3]dioxolane (7.7 mg, 0.030 mmol) in acetonitrile (1 ml). The reaction mixture was heated to 70 degrees for 30 minutes. The crude product was purified by reverse phase preparative chromatography (Vydac 218TP1022 column; solvents A=water/0.1% TFA and B=CH₃CN/0.1% TFA; gradient 40-80% B over 40 min; flow 10 ml/minute; detection at 254 nm). The fractions were left in the fridge overnight and to the acetonitrile phase was added diethyl ether, dried (Na₂SO₄) and evaporated under reduced pressure. (Analytical HPLC: Vydac 218TP54 column; solvents: A=water/0.1% TFA and B=CH₃CN/0.1% TFA; gradient 40-80% B over 20 min; flow 1.0 ml/minute; retention time 9.200 minutes detected at 214 and 254 nm).

Structure verified by NMR.

The protecting group on 3-(2-fluoromethyl-phenylsulfanyl)-propionaldehyde (0.81 mg, 0.0034 mmol) was removed using 1N HCl in acetonitrile (1:1) 0.1 ml for 30 minutes.

### Example 2

### Synthesis of (2-formylethyl)-4-fluorobenzamide

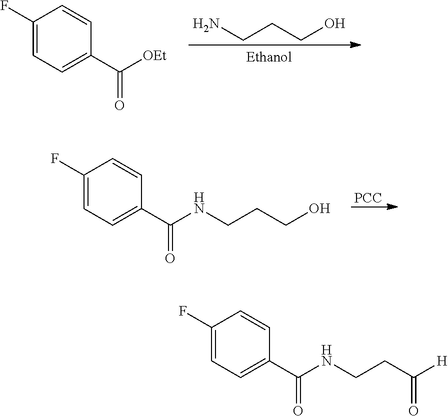

### 2a. Preparation of (3-hydroxypropyl)-4-fluorobenzamide

To a dry 100 ml 3 necked round bottomed flask (RBF) provided with nitrogen, 5.68 g (0.07562 mole) of 3-amino-1-propanol, 12.68 g of TEA in 100 ml dry ethyl acetate was added and cooled to 0-5° C. 4-fluorobenzoyl chloride (10 g, 0.0630 mole) in ethyl acetate was then added drop-wise over a period of 30 min and allowed stir overnight. Progress of the reaction was monitored by thin layer chromatography (TLC). After the completion of the reaction, ethyl acetate was distilled out completely and the residue extracted again with ethylacetate/washed with water dilute sodium bicarbonate solution and dried. Ethyl acetate layer was then distilled and the residue was purified by silica column using methanol dichloromethane (5-20%) as eluent. Yield: 5.86 g (50%); Purity: 93.9%; ¹H-NMR (CDCl₃): 3.6 (d, 2H, CH₂), 3.8 (d, 2H, CH₂), 7.01 (s, 1H, NH), 7.1 (d, 2H, ArH), 7.8 (d, 2H, ArH); MS: 198 (M+1)

### 2b. Preparation of (2-formylethyl)-4-fluorobenzamide

To a dry 50 ml 3 necked RBF provided with nitrogen, 3.2 g of PCC (0.0148 mole) and 2.0 g of silica gel in 32 ml dry dichloromethane was added and cooled to −5 to −10° C. 2.0 g

(0.01014 mole) of (3-hydroxypropyl)-4-fluorobenzamide in dichloromethane was then added drop-wise over a period of 30 min and allowed stir overnight at RT. Progress of the reaction was monitored TLC. After the completion of the reaction, dichloromethane was distilled out completely and the residue residue was purified by combiflash using silica column twice. Eluent used was 0-10% methanol in dichloromethane. Yield: 0.2 g (10%); Purity: 89%; ¹H-NMR (CDCl₃): 2.8 (d, 2H, CH₂), 3.8 (d, 2H, CH₂), 6.8 (s, 1H, NH), 7.1 (d, 2H, ArH), 7.8 (d, 2H, ArH); 10.0 (s, 1H, CHO) MS: 314 (M+1)

### Example 3

### Synthesis of 6-(3-fluororpropyloxy)-2-naphthaldehyde

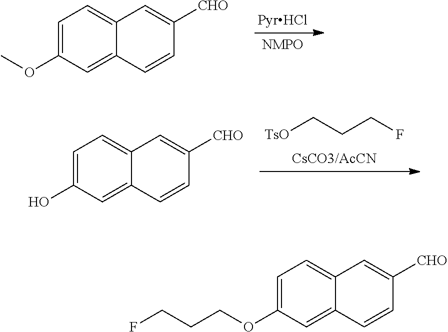 [SMILES: COc1ccc2cc(C=O)ccc2c1 ; Cc1ccc(S(=O)(=O)OCCCF)cc1 ; O=Cc1ccc2cc(O)ccc2c1 ; O=Cc1ccc2cc(OCCCF)ccc2c1]

### 3a. Preparation of 6-Hydroxy-2-naphthaldehyde

In 25 ml single neck RBF 6-methoxy-2-naphthaldehyde (0.5 g, 0.00268 mole), pyridine hydrochloride (1.24 g, 0.0107 mole) in 5 ml NMPO was heated at 110° C. for 24 h. Progress of the reaction was monitored by TLC. Reaction mixture was then cooled and diluted with water. The product was extracted to ethyl acetate, dried over anhydrous sodium sulphate and distilled. The crude product was then purified through silica gel column using dichloromethane and methanol (1-5%) as eluent. Yield: 0.23 g; Purity: 99.8%; ¹H-NMR (CDCl₃): 7.25 (dd, 2H, ArH), 7.7 (d, 1H, ArH), 7.8 (dd, 2H, ArH), 8.3 (d, 1H, ArH), 10.1 (s, 1H, CHO); MS: 173 (M+1)

### 3b. Preparation of 6-(3-fluoropropyloxy)-2-naphthaldehyde

In 25 ml two neck RBF 6-hydroxy-2-naphthaldehyde (0.1 g, 0.00058 mole), cesium carbonate (0.22 g, 0.0012 mole) in 5 ml acetonitrile added with fluoropropyl tosylate (0.140 g, 0.00060 mole) and refluxed for 10 h. Progress of the reaction was monitored by TLC. After the completion of the reaction, cateonitrile was distilled out and the product was extracted to ethyl acetate, dried over anhydrous sodium sulphate and distilled. The crude product was then purified through silica gel column using dichloromethane and methanol (1-5%) as eluent, Yield: 0.1 g; HPLC Purity: 98.2%; ¹H-NMR (CDCl₃): 4.2-4.8 (m, 6H, 3×CH₂), 7.7 (d, 1H, ArH), 7.8 (dd, 2H, ArH), 8.3 (d, 1H, ArH), 10.1 (s, 1H, CHO); MS: 233 (M+1)

### Example 3A

### Synthesis of 6-(2-fluoroethyloxy)-2-naphthaldehyde (GEH120143)

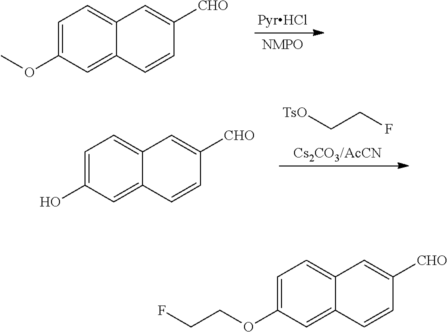 [SMILES: COc1ccc2cc(C=O)ccc2c1 ; Cc1ccc(S(=O)(=O)OCCF)cc1 ; O=Cc1ccc2cc(O)ccc2c1 ; O=Cc1ccc2cc(OCCF)ccc2c1]

### 3A.a. Preparation of 6-hydroxy-2-naphthaldehyde

6-Hydroxy-2-naphtaldehyde was prepared as described in example 3.

### 3A.b. Preparation of fluoroethyl tosylate

2-Fluoroethanol (50.7 g, 792 mmol) was dissolved in pyridine (350 mL) and the solution cooled in an ice-salt bath. Tosyl chloride (151 g, 792 mmol) was added in portions over approximately 30 min keeping the temperature below 5° C. The mixture was stirred for 4 h at 0° C., quenched with ice cooled water (600 mL) and extracted with ethyl acetate (3×250 mL). The combined organic extracts were washed with hydrochloric acid (1 M) until the aqueous phase remained acidic, followed by washing with potassium carbonate (10%, 2×200 mL) and brine. The organic phase was dried (magnesium sulphate), filtered and concentrated, giving an almost colourless oil (72.6 g, 42%). ¹H NMR (300 MHz, CDCl₃): δ 2.45 (3H, s, CH₃), 4.33 (2H, dt, J=13.5 Hz, 4.0 Hz, OCH₂), 4.57 (2H, dt, J=47.0 Hz, 4.0 Hz, CH₂F), 7.35 (2H, d, J=8.0 Hz, Ar), 7.80 (2H, d, J=8.0 Hz, Ar). ¹³C NMR (75 MHz, CDCl₃): δ 21.6 (CH₃), 68.7 (d, J=19.0 Hz, OCH₂), 80.5 (d, J=172.0 Hz, CH₂F), 128.0 (Ar), 129.9 (Ar), 132.6 (CMe), 145.1 (C—S). ¹⁹F NMR (CDCl₃): δ −224.5.

### 3A.c. Preparation of 6-(2-fluoroethyloxy)-2-naphthaldehyde

A solution of 6-hydroxy-2-naphthaldehyde (0.04 g, 0.23 mmol), cesium carbonate (0.088 g, 0.46 mmol) and fluoroethyl tosylate (0.061 g, 0.276 mmol) in acetonitrile (2 mL) was refluxed for 10 h. Progress of the reaction was monitored by TLC. The reaction mixture was concentrated and the residue was taken up in ethyl acetate, dried over anhydrous sodium sulphate and concentrated. The crude product was filtered through silica gel column using 5% methanol in dichloromethane, concentrated and purified by preparative HPLC (column Phenomenex Luna C18 (2) 21.20×250 mm, 5 μm, flow 10 mL/min, solvents A: water/0.1% TFA and B: acetonitrile/0.1% TFA, gradient 10 to 80% B over 60 min, UV detection at 214 nm), yielding 14 mg after lyophilisation. ¹H NMR (500 MHz, CDCl₃): δ 4.38 (2H, m, ³J_{FH}=27 Hz, CH₂O), 4.85 (2H, m, ²J_{FH}=47 Hz, FCH₂), 7.19 (1H, m, Ar), 7.29 (1H, m, Ar), 7.81 (1H, m, Ar), 7.92 (1H, m, Ar), 7.93 (1H, m, Ar), 8.27 (1H, m, Ar), 10.11 (1H, s, CHO). ¹³C NMR (125 MHz, CDCl₃): δ 67.3 (d, ²J_{FC}=21.0 Hz, CH₂O), 81.7 (d, ¹J_{FC}=170.9 Hz, CH₂F), 107.0 (ArH), 120.1 (ArH), 123.7 (ArH), 127.8 (ArH), 128.2 (Ar), 131.4 (ArH), 132.6 (ArCHO), 134.2 (ArH), 138.1 (Ar), 159.0 (ArO), 192.0 (CHO).

### Example 4

### Synthesis of 5-Iodo-6-methoxy-naphthalene-2-carbaldehyde

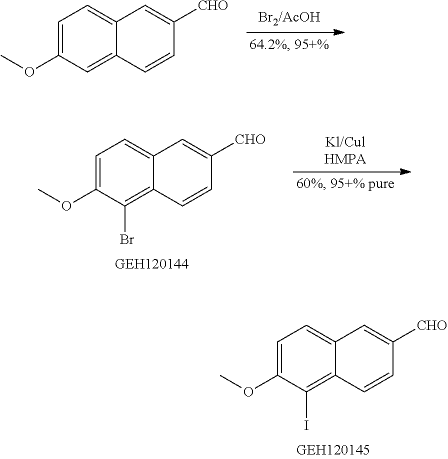

### 4a. Preparation of 5-Bromo-6-methoxy-naphthalene-2-carbaldehyde

Bromine (556 μL, 10.8 mL) in 10 mL of glacial HOAc was added under nitrogen dropwise over 1 h to a solution of 6-methoxy-naphthalene-2-carbaldehyde (2.01 g, 10.8 mmol) in 25 mL of glacial HOAc at room temperature. After the addition the reaction was stirred at room temperature for 2 h. The solid was collected by filtration, rinsed with glacial HOAc and dried under reduced pressure to give 5-bromo-6-methoxy-naphthalene-2-carbaldehyde (2.27 g, 79%) as a light pink solid, HPLC Purity: 99.5%; ¹H-NMR (CDCl₃): 4.2 (s, 3H, OCH₃), 7.8 (d, 1H, ArH), 8.0 (dd, 2H, ArH), 8.3 (dd, 2H, ArH), 10.1 (s, 1H, CHO); MS: 265.1 (M+1)

### 4b. Preparation of 5-Iodo-6-methoxy-naphthalene-2-carbaldehyde

5-Bromo-6-methoxy-naphthalene-2-carbaldehyde (0.5 g, 0.00188 mol) in 6.25 ml of HMPA was added copper iodide (1.79 g, 0.0094 mol) and potassium Iodide (0.0188 mol) and heated to 160° C. Reaction mixture was maintained for ˜20 h and then quenched by adding dilute HCl. The solid obtained is filtered and purified through silica gel column with Hexane ethyl acetate as eluent. Yield: 0.1 g; HPLC Purity:92.1%; ¹H-NMR (CDCl₃): 4.2 (s, 3H, OCH₃), 7.8 (d, 1H, ArH), 8.0 (dd, 2H, ArH), 8.3 (dd, 2H, ArH), 10.1 (s, 1H, CHO); MS: 313 (M+1)

### Example 5

### General Preparation of Internal Carboxylic Acid Standards

Internal standards such as carboxylic acids are synthesized using Oxone.

### 5a. General Procedure

Aldehyde (0.002 mole) is taken in dimethylformamide (DMF) and OXONE (0.24 mole) was added to it and the reaction mixture was stirred overnight. Progress of the reaction was monitored using TLC. Distilled water was then added and the solid obtained was filtered.

### 5b. Purification

The solid was then purified by dissolving first bicarbonate, extracting out the organic impurities and then re-precipitating with dilute hydrochloric acid at pH 2.0-3.0. All the compounds are isolated with a purity of 95+% by HPLC analysis.

### Example 6

### Screening for ALDH Activity

### 6a. ALDH Assay

Aldehyde Dehydrogenase is an enzyme that acts on aldehydes as substrates and converts them to acid (products).

### Principle:

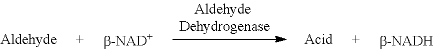

Abbreviations used:

β-NAD⁺=β-Nicotinamide Adenine Dinucleotide, Oxidized Form

β-NADH=β-Nicotinamide Adenine Dinucleotide, Reduced Form

Designing and Standardization of ALDH Assay:
following the conversion of NAD+ to NADH typically one does the ALDH assays.

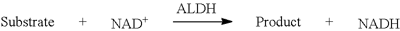

The formation of NADH is monitored by measuring the absorbance at 340 nm. However, before employing this method, the compounds were screened for their spectral properties, especially to avoid any interference in absorbance either from the substrate or the product.

Spectral Studies of the Compounds:
Absorbance Spectra: The compounds were initially screened for their absorbance from 200 nm to 800 nm.
Fluorescence Spectra: In some cases, the studies indicated that the compounds (Substrate or products) had interfering absorbance at 340 nm. Such compounds were further screened for their fluorescence properties by recording their excitation/emission wavelengths.

ALDH Assay by Spectroscopic Method:
The ALDH assay is designed to measure either the utilization of the substrate or formation of product by measuring at their unique wavelengths (Absorbance or Fluorescence).

### 6b. Spectral Studies

All the spectral studies for the compounds were carried out in 0.1M Tris HCl pH 8.0 buffer. CSCT Compounds were initially dissolved in Methanol (˜2.0 mg/mL). The compounds were further diluted in 0.1M Tris HCl pH 8.0 buffer (concentration ranging from ˜20 to 50 μg/mL). The Spectra was recorded using Spectramax M5.

The ALDH activity can be followed either by monitoring the conversion of β-NAD⁺ to β-NADH or by directly monitoring the product/substrate. The conversion of β-NAD⁺ to β-NADH yields increasing in absorbance at 340 nm. If either the substrate/products have any spectral interference at this wavelength then unique absorbance/fluorescence wavelength of either product/substrate are used. The measurements were taken on Spectromax M5.

### 6c. ALDH Assay Reagents

1. Reagent 1: 1 M Tris HCl Buffer, pH 8.0 at 25° C. (Prepare 50 ml in deionized water using Trizma Base, Sigma Prod. No. T-1503. Adjust to pH 8.0 at 25° C. with 1 M HCl.)
2. Reagent 2: 20 mM β-Nicotinamide Adenine Dinucleotide, Oxidized Form, Solution (β-NAD⁺) (Prepare 1 ml in deionized water using β-Nicotinamide Adenine Dinucleotide, PREPARE FRESH).
3. Reagent 3: 3 M Potassium Chloride Solution (KCl) (Prepare 1 ml in deionized water using Potassium Chloride).
4. Reagent 4: 1 M 2-Mercaptoethanol Solution (2-ME) (Prepare 1 ml in deionized water using 2-Mercaptoethanol. PREPARE FRESH.)
5. Reagent 5: 100 mM Tris HCl Buffer with 0.02% (w/v) Bovine Serum Albumin, pH 8.0 at 25° C. (for Enzyme Dilution).
6. Reagent 6: Aldehyde Dehydrogenase Enzyme Solution (Yeast ALDH). Immediately before use, prepare a solution containing 0.5-1 unit/ml of Aldehyde Dehydrogenase in cold Reagent 5).

### 6d. ALDH Assay Method

Pipette (in milliliters) the following reagents into vial:

| | Test | Blank |
|---|---|---|
| Deionized Water | 2.32 | 2.32 |
| Reagent 1 (Buffer) | 0.30 | 0.30 |
| Reagent 2 (β-NAD) | 0.10 | 0.10 |
| Reagent 3 (KCl) | 0.10 | 0.10 |
| Reagent 7 (Substrate) | 0.05 | 0.05 |
| Reagent 4 (2-ME) | 0.03 | 0.03 |
| Mix by inversion and equilibrate to 25° C. | | |
| Reagent 5 (Enz Dil) | — | 0.10 |
| Reagent 6 (Enzyme Solution) | 0.10 | — |

**Reagent 7 (Substrate): 50 μM concentration of Substrate in 0.1M TrisHCl pH 8.0 buffer.

### 6e. Final Assay Concentration

In a 3.00 ml reaction mix, the final concentrations are 103 mM Tris HCl Buffer (Reagent 1), 0.67 mM β-nicotinamide adenine dinucleotide (Reagent 2), 100 mM potassium chloride (Reagent 3), 10 mM 2-mercaptoethanol (Reagent 4), 0.0007% (w/v) bovine serum albumin (Reagent 5) and 0.05-0.1 unit aldehyde dehydrogenase (Reagent 6).

TABLE 1
Substrates selected for ALDH assay

| | | Commercial/ | Log P |
|---|---|---|---|
| Compound code | Structure | synthesized | (clogP) |
| 4-fluorobenzaldehyde | 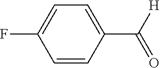 | Commercial | 1.8 |
| Example 2 | 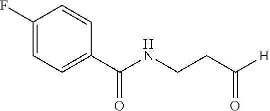 [SMILES: O=CCCNC(=O)c1ccc(F)cc1] | Synthesized | 0.63 |
| Example 3 | 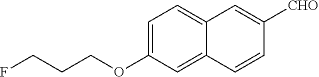 [SMILES: O=Cc1ccc2cc(OCCCF)ccc2c1] | Synthesized | 2.95 |
| Example 3A | 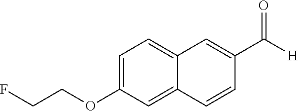 [SMILES: O=Cc1ccc2cc(OCCF)ccc2c1] | Synthesized | 3.2 |
| Example 4 | 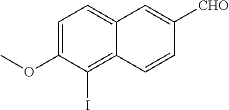 | Synthesized | 4.01 |
| 4-Iodobenzaldehyde | 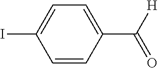 | Commercial | 3.14 |
| 6-Methoxy-2- Naphthaldehyde | 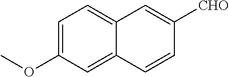 | Commercial | 2.65 |
| 2-Naphthaldehyde | 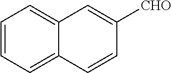 [SMILES: O=Cc1ccc2ccccc2c1] | Commercial | 2.78 |
| 3-anisladehyde | 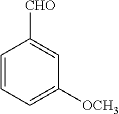 | Commercial | 1.65 |
| 4-(N,N-diethylamino) benzaldehyde | 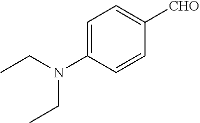 | Commercial | 2.74 |
| ALDEFLUOR® | 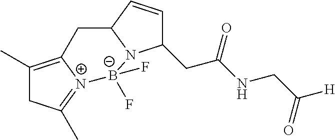 [SMILES: CC1=C2CC3C=CC(CC(=O)NCC=O)N3[B-](F)(F)[N+]2=C(C)C1] | Stem cell technologies | NA |

### 6 Results

The results of the ALDH assay are summarized in Table 2.

TABLE 2
Screening results:

| | | Commercial/ | Log P | |
|---|---|---|---|---|
| Compound | Structure | synthesized | (clog P) | Comments |
| 4- fluorobenzaldehyde | 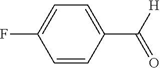 | Commercial | 1.8 | Active |
| Example 2 | 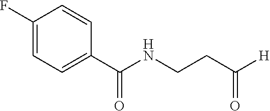 [SMILES: O=CCCNC(=O)c1ccc(F)cc1] | Synthesized | 0.63 | Not active |
| Example 3 | 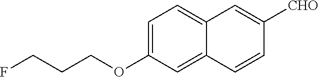 [SMILES: O=Cc1ccc2cc(OCCCF)ccc2c1] | Synthesized | 2.95 | Active |
| Example 3A | 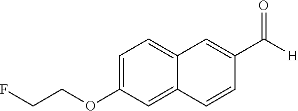 [SMILES: O=Cc1ccc2cc(OCCF)ccc2c1] | Synthesized | 3.2 | Active |
| Example 4 | 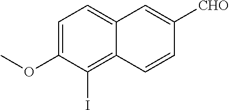 | Synthesized | 4.01 | Due to spectral interference, ALDH assay cannot be designed by spectroscopic methods, HPLC method is recommended. |
| 4- Iodobenzaldehyde | 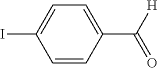 | Commercial | 3.14 | Active |
| 6-Methoxy-2- Naphthaldehyde | 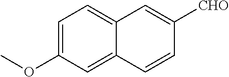 | Commercial | 2.65 | Active |
| 2-Naphthaldehyde | 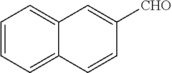 [SMILES: O=Cc1ccc2ccccc2c1] | Commercial | 2.78 | Active |
| 3-anisladehyde | 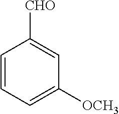 | Commercial | 1.65 | Active |
| 4-(N,N-diethyl) benzaldehyde | 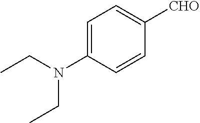 | Commercial | 2.74 | Due to spectral interference, ALDH assay cannot be designed by spectroscopic methods, HPLC method is recommended. |
| ALDEFLUOR® | 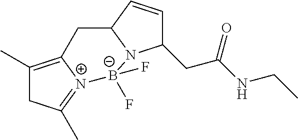 [SMILES: CCNC(=O)CC1C=CC2CC3=C(C)CC(C)=[N+]3[B-](F)(F)N12] | Stem cell technologies | NA | Active |

Active: Compounds for which enzymatic activity was observed spectroscopically either by change in absorbance or fluorescence as a function of time.
Non active: Compounds for which no enzymatic activity was observed spectroscopically either by change in absorbance or fluorescence as a function of time.

### Example 7

### General Radiosynthesis Method for Preparation of ¹⁸F-Compounds

¹⁸F-fluoride (up to 370 MBq) is azeotropically dried in the presence of Kryptofix 222 (12-14 mg in 0.5 ml MeCN) and potassium carbonate (100 μl 0.1M solution in water) by heating under N₂ to 125° C. for 15 mins. During this time 2×1 ml MeCN are added and evaporated. After cooling to <40° C., a solution of precursor compound such as trimethylammonium benzaldehyde triflate (3-7 mg in 0.7 ml DMSO) is added. The reaction vessel is sealed and heated to 120° C. for 15 mins to effect labelling. The crude reaction mixture is cooled to room temperature and diluted by addition to 10 ml water. The mixture is passed sequentially through a Sep-pak CM-plus cartridge (conditioned with 10 ml water) and a SepPak C18-plus cartridge (conditioned with 20 ml EtOH and 20 ml H₂O). The cartridges are flushed with water (10 ml), and the product, such as ¹⁸F-fluorobenzaldehyde is eluted from the SepPak C18-plus cartridge with MeOH (1 ml).

### Example 8

### Cell Based ALDH Assay for 6-(2-fluoroethyloxy)-2-naphthaldehyde

### Summary—

Briefly, the compound was dissolved in DMSO and competed against ALDEFLUOR™, a BODIPY-conjugated ALDH substrate, in a cell-based assay using SK-BR-3 cells. The BODIPY fluorescence in the cell samples were measured using FACS at 488 nm. The median fluorescence of each sample was measured and fitted to a sigmoidal dose-response curve for calculation of IC₅₀ using Prism Graphpad. The results demonstrate a decrease in fluorescence of the samples with increasing concentrations of the tested compound, this suggests that the compound is ALDH substrates and can displace ALDEFLUOR™. The IC₅₀ value was 330 nM.

### Method and Material

Test Compounds—

6-(2-fluoroethyloxy)-2-naphthaldehyde was dissolved and diluted in DMSO prior to use.

Cell Line—

SK-BR-3 cells, a cell line reported to have a high expression of ALDH+ cells, was used for all experiments. The cells were cultured in RPMI media supplemented with 10% fetal bovine serum and 2 mM L-glutamine, in 37° C., 5% CO₂. On the day of assay, cells were harvested by trypsination, centrifuged, and re-suspended in ALDEFLUOR™ assay buffer to a concentration of 1×10⁶ cells.

Competition Assay—

The compound was competed against ALDEFLUOR™, a BODIPY-conjugated ALDH substrate. Two series of cell samples with a fixed concentration of ALDEFLUOR™ were prepared according to the manufacturers protocol (ALDEFLUOR™ kit #01700, Stem Cell Technologies), either with or without addition of the inhibitor DEAB. The compound was added to the cell samples for a final concentration of 0.005-50 μM. Following incubation at 37° C., the fluorescence was measured in each sample by FACS at 488 nm. The assay was repeated in triplicate.

The median fluorescence of each sample was calculated, and the values were then normalized and fitted to a dose-response curve for calculation of IC₅₀ using Prism Graphpad.

### Results

6-(2-fluoroethyloxy)-2-naphthaldehyde displaced ALDEFLUOR™, as is demonstrated by the decrease in fluorescence of the samples with increasing compound concentration. Calculation of IC₅₀ values result in an IC₅₀ of 330 nM (Error! Reference source not found.). The results suggest that the compound is a potent ALDH substrate, and can efficiently displace ALDEFLUOR™ in vitro.

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

1. A method for detection of tumour stem cells in a subject, comprising:
(i) administrating a detectably labelled substrate for ALDH to said subject;
(ii) detecting uptake of said detectably labelled substrate for ALDH by in vivo imaging;
wherein the detectably labelled substrate for ALDH is 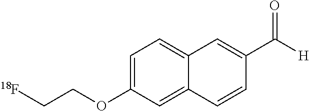 [SMILES: O=Cc1ccc2cc(OCC[18F])ccc2c1]
or a salt or solvate thereof.

2. The method of claim 1, further comprising identifying ALDH expressing cells within a tumor.

3. A method for detection of tumor stem cells in a subject, comprising:
(i) administrating to said subject a compound 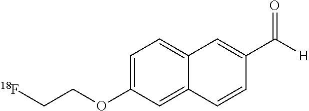 [SMILES: O=Cc1ccc2cc(OCC[18F])ccc2c1]
or a salt or solvate thereof;
(ii) detecting uptake of said compound by in vivo radioimaging.

4. A method of monitoring the effect of treatment of a tumour in a subject, said method comprising
(i) administrating a detectably labelled substrate for ALDH to said subject;
(ii) detecting uptake of said detectably labelled substrate for ALDH by in vivo imaging;
wherein the detectably labelled substrate for ALDH is 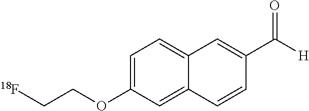 [SMILES: O=Cc1ccc2cc(OCC[18F])ccc2c1]
or a salt or solvate thereof,
said method being effected optionally before, during and after treatment.

5. A method for radiotherapy of a cancer patient, comprising administration of an effective amount of radiotherapy-labelled substrate for ALDH to said cancer patient wherein the radiotherapy-labelled substrate for ALDH is 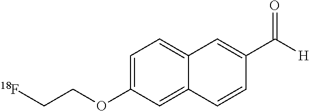 [SMILES: O=Cc1ccc2cc(OCC[18F])ccc2c1]
or a salt or solvate thereof.

6. A compound of formula 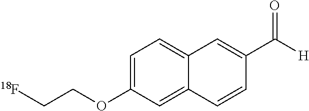 [SMILES: O=Cc1ccc2cc(OCC[18F])ccc2c1]
or a salt or solvate of any thereof, for use in medicine.

7. A pharmaceutical formulation comprising the compound of claim 6 and a pharmaceutically acceptable excipient.
